(19) United States
(12) Patent Application Publication
(10) Pub. No.: US 2025/0198996 A1
(43) Pub. Date: Jun. 19, 2025

(54) EXTENDED LIVE CELL BARCODING APPROACH FOR MULTIPLEXED MASS CYTOMETRY

(71) Applicant: BOARD OF REGENTS, THE UNIVERSITY OF TEXAS SYSTEM, Austin, TX (US)

(72) Inventors: Michael ANDREEFF, Houston, TX (US); Muharrem MUFTUOGLU, Houston, TX (US)

(73) Assignee: BOARD OF REGENTS, THE UNIVERSITY OF TEXAS SYSTEM, Austin, TX (US)

(21) Appl. No.: 18/867,701

(22) PCT Filed: May 19, 2023
(86) PCT No.: PCT/US2023/022952

§ 371 Date: Nov. 20, 2024

Related U.S. Application Data
(60) Provisional application No. 63/344,211, filed on May 20, 2022.

(51) Int. Cl. G01N 33/532; G01N 1/30; G01N 15/10; G01N 33/569; G01N 33/577

(52) U.S. Cl. CPC G01N 33/532; G01N 1/30; G01N 15/10; G01N 33/569; G01N 33/577; G01N 2015/1006; G01N 2458/15

(57) ABSTRACT

Provided are novel metal tags, metal-tagged probes and methods of making and using them for barcoding and mass cytometry. Also provided are kits comprising metal-tagged probes.

### CROSS-REFERENCE TO RELATED APPLICATIONS

This application claims the benefit of priority to U.S. Provisional Application No. 63/344,211, filed May 20, 2022, the contents of which are incorporated herein by reference in its entirety.

### BACKGROUND

Mass cytometry, also known as cytometry by time-of-flight (CyTOF) is useful for phenotypic and functional analysis at the single-cell level. For CyTOF, monomer or polymer metal chelators are loaded with metal isotopes and conjugated to antibodies. Due to mass differences of isotopes used in metal tagged antibodies, multiple metal-tagged antibody labeled samples can be processed together thereby improving sample-to-sample consistency and overcoming technical variations.

The number of analytes measured by CyTOF relies on, among other things, isotope purification, design of novel chelators to capture different classes of metals, and the availability of appropriate chemical methods for antibody labeling. The mass window of current mass cytometers theoretically allows assessment of 135 parameters per cell, however, reagents to conduct such comprehensive analyses are not currently available. In addition, currently the availability of barcoding reagents for live-cell barcoding is limited due to limited sensitivity achieved with some metal isotopes and limits on the number of polymer-metal tag combinations available.

Therefore, there exists a need for additional barcoding reagents to expand the number of samples that can be analyzed simultaneously using live-cell barcoding schemes.

### BRIEF SUMMARY

Provided are compositions of isotopes and polymers useful for elemental analysis using mass cytometry (CyTOF). The compositions comprise populations of metal tags, wherein each metal tag comprises a polymer and an isotope.

In some aspects, the polymer comprises at least one metal-chelating group. In some aspects, the polymer is selected from X8 polymer and metal-chelating polymer 9 (MCP9).

In some aspects, the isotope has an atomic mass between about 89 and about 208. In some aspects, the composition comprises at least one metal tag that comprises 103Rh.

In some aspects, the polymer of the population of metal tags is MCP9.

In some aspects, the isotope is selected from cadmium, palladium, indium, yttrium, rhodium, and combinations thereof.

In some aspects, in addition to the at least one metal tag comprising 103Rh, the population of metal tags comprises one or more isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, 103Rh, and 115In.

In some aspects, in addition to the at least one metal tag comprising 103Rh, the population of metal tags comprises at least four isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, 103Rh, and 115In.

In some aspects, in addition to the at least one metal tag comprising 103Rh, the population of metal tags comprises at least six isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, 103Rh, and 115In.

In some aspects, in addition to the at least one metal tag comprising 103Rh, the population of metal tags comprises at least eight isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, 103Rh, and 115In.

In some aspects, in addition to the at least one metal tag comprising 103Rh, the population of metal tags comprises at least ten isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, 103Rh, and 115In.

In some aspects, in addition to the at least one metal tag comprising 103Rh, the population of metal tags comprises at least twelve isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, 103Rh, and 115In.

In some aspects, in addition to the at least one metal tag comprising 103Rh, the population of metal tags comprises at least fourteen isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, 103Rh, and 115In.

In some aspects, in addition to the at least one metal tag comprising 103Rh, the population of metal tags comprises 106Cd, 110Cd, 111Cd, 112Cd, 113Cd, 114Cd, 116Cd, 102Pd, 104Pd, 105Pd, 106Pd, 108Pd, 110Pd, 89Y, 103Rh, and 115In.

In some aspects, the population of metal tags comprises 106Cd, 110Cd, 111Cd, 112Cd, 113Cd, 114Cd, 116Cd, 102Pd, 104Pd, 105Pd, 108Pd, 89Y, 103Rh, and 115In.

Also provided are populations of metal tags, wherein each metal tag comprises a polymer comprising at least one metal-chelating group, and an isotope, wherein at least one metal tag comprises an isotope selected from 185Re and 187Re.

In some aspects, the polymer of the metal tag is selected from X8 polymer and metal-chelating polymer 9 (MCP9). In some aspects, the population of metal tags further comprises at least one isotope that has an atomic mass between about 89 and about 208. In some aspects, the at least one isotope having an atomic mass between about 89 and about 208 is selected from yttrium, ruthenium, rhodium, tin, lanthanum, mercury and lead.

In some aspects, in addition to the at least one metal tag comprising an isotope selected from 185Re and 187Re, the population of metal tags comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least one metal tag of the population of metal tags comprising an isotope selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In and at least one metal tag of the population of metal tags comprising an isotope selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least six isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least ten isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least twelve isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least fourteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least sixteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least eighteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least twenty isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least twenty-two isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least twenty-four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least twenty-six isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least twenty-eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least twenty-nine isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least thirty isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises at least thirty-one isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, in addition to the at least one metal tag comprising an isotope selected from 185Re and 187Re, the population of metal tags comprises 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

Also provided are populations of metal tags, wherein each metal tag comprises a polymer comprising at least one metal-chelating group, and an isotope, wherein at least one metal tag comprises an isotope selected from 196Hg, 198Hg, 199Hg, 200Hg, 201Hg, 202Hg, and 204Hg.

In some aspects, the polymer of the metal tag is selected from X8 polymer and metal-chelating polymer 9 (MCP9). In some aspects, the population of metal tags further comprises at least one isotope that has an atomic mass between about 89 and about 208. In some aspects, the at least one isotope having an atomic mass between about 89 and about 208 is selected from yttrium, ruthenium, rhodium, tin, lanthanum, lead, and rhenium.

In some aspects, in addition to the at least one metal tag comprising an isotope selected from 196Hg, 198Hg, 199Hg, 200Hg, 201Hg, 202Hg, and 204Hg, the population of metal tags comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least one metal tag of the population of metal tags comprising an isotope selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In and at least one metal tag of the population of metal tags comprising an isotope selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least one metal tag of the population of metal tags comprising an isotope selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb and at least one metal tag of the population of metal tags comprising an isotope selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least six isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb. 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least ten isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least twelve isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least fourteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least sixteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least eighteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least twenty isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least twenty-two isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises at least twenty-four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

In some aspects, in addition to the at least one metal tag comprising an isotope selected from 196Hg, 198Hg, 199Hg, 200Hg, 201Hg, 202Hg, and 204Hg, the population of metal tags comprises 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

Further provided are populations of metal tags, wherein each metal tag comprises a polymer comprising at least one metal-chelating group, and an isotope, wherein at least one metal tag comprises an isotope selected from 204Pb, 206Pb, 207Pb, and 208Pb. In some aspects, the polymer of the metal tag is selected from X8 polymer and metal-chelating polymer 9 (MCP9). In some aspects, the population of metal tags further comprises at least one isotope that has an atomic mass between about 89 and about 208.

In some aspects, the at least one isotope having an atomic mass between about 89 and about 208 is selected from yttrium, ruthenium, rhodium, tin, lanthanum, mercury, and rhenium.

In some aspects, in addition to the at least one metal tag comprising an isotope selected from 204Pb, 206Pb, 207Pb, and 208Pb, the population of metal tags comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least one metal tag of the population of metal tags comprising an isotope selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In and at least one metal tag of the population of metal tags comprising an isotope selected 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re

In some aspects, in addition to the at least one metal tag comprising an isotope selected from 204Pb, 206Pb, 207Pb, and 208Pb, the population of metal tags comprises at least one metal tag of the population of metal tags comprising an isotope selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb and at least one metal tag of the population of metal tags comprising an isotope selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least one metal tag of the population of metal tags comprising an isotope selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re and at least one metal tag of the population of metal tags comprising an isotope selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least six isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least ten isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least twelve isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least fourteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least sixteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least eighteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least twenty isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least twenty-two isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least twenty-four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least twenty-six isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least twenty-eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises at least twenty-nine isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, in addition to the at least one metal tag comprising an isotope selected from 204Pb, 206Pb, 207Pb, and 208Pb, the population of metal tags comprises 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

Further provided are populations of metal tags, wherein each metal tag comprises a polymer comprising at least one metal-chelating group, and an isotope, wherein at least one metal tag comprises 139La. In some aspects, the polymer of the metal tag is selected from X8 polymer and metal-chelating polymer 9 (MCP9). In some aspects, the population of metal tags further comprises at least one isotope that has an atomic mass between about 89 and about 208. In some aspects, the at least one isotope having an atomic mass between about 89 and about 208 is selected from yttrium, ruthenium, rhodium, tin, mercury, lead, rhenium, and combinations thereof.

In some aspects, in addition to the at least one metal tag comprising 139La, the population of metal tags comprises at least four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least six isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least ten isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags of comprises at least twelve isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least fourteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least sixteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least eighteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal comprises at least twenty isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least twenty-two isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least twenty-four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least twenty-six isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least twenty-eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least twenty-nine isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises at least thirty isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, in addition to the at least one metal tag comprising 139La, the population of metal tags comprises 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

In some aspects, provided is a population of metal tags that comprises at least one metal tag that comprises one or more isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In and at least one metal tag that comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, and 208Pb.

In some aspects, provided is a population of metal tags that comprises at least one metal tag that comprises one or more isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In and at least one metal tag that comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, provided is a population of metal tags that comprises at least one metal tag that comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, and 208Pb and at least one metal tag that comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, the population of metal tags comprises a polymer that is X8 polymer and one or more isotopes selected from 96Ru, 98Ru, 99Ru, 100Ru, 101Ru, 102Ru, and 104Ru.

In some aspects, the population of metal tags comprises isotopes 103Rh, 89Y, and 139La.

Also provided are populations of metal-tagged probes, wherein each metal-tagged probe comprises a metal tag of a population of metal tags as described herein and a biomolecule.

In some aspects, the metal tag is conjugated to the biomolecule.

In some aspects, the biomolecule is a protein. In some aspects, the protein is an antibody. In some aspects, the protein and/or antibody has at least one sulfhydryl group and the metal tag is conjugated to the at least one sulfhydryl group.

In some aspects, the antibody is selected from an anti-CD45, anti-CD19, anti-CD3, anti-CD4, anti-CD8, anti-TNFα, anti-IFN-γ, anti-CD16, anti-CD33, anti-CD36, anti-phosphoSTAT5, anti-IL2, anti-CD28, anti-CD14, anti-T-bet, anti-CD66b, anti-B2M, anti-CD20, anti-HLA-DR, anti-CD14, anti-CD56, anti-CD294, anti-CD38, anti-CD11c, anti-CD57, anti-CD123, anti-TCRγδ, anti-CD161, anti-CCR7, anti-CD45RA, anti-CD27, anti-CD45RO, anti-CD25, anti-CCR5, anti-CCR4, anti-CXCR3, and anti-CCR6, anti-CD26, anti-KG2A, anti-CD49F, anti-CD127, anti-CCR2, anti-TIGIT, anti-Ki-67, anti-TIM3, anti-2B4, anti-ICOS, anti-PD-1, anti-Eomes, anti-CD137, anti-CTLA-4, anti-LAG3, anti-Perforin, anti-CD160, anti-CD69, anti-CD39, anti-KLRG1, anti-CD95, anti-GITR, anti-CLA, anti-CD73, Anti-CD298, anti-CD98, anti-G6PD, anti-GAPDH, anti-CD15S, anti-pS6, anti-pAKT, anti-MEK1/2, anti-pERK1/2, anti-phosphoSTAT3, anti-phosphoSTAT1, anti-phosphoSTAT4, anti-phosphoSTAT6, anti-p4EBP1, anti-GLUT1, anti-H3K27, anti-c-myc, anti-granzyme A, anti-granzyme B, anti-granzyme K, anti-granzyme M, anti-Helios, anti-RUNX3, anti-p53, anti-HIF1a, anti-p-NRF2, anti-Bcl-2, anti-Mcl-1, anti-Bcl-xL, anti-BIM, anti-Bax, anti-CD90, anti-CD96, anti-OX40, anti-MIP1a, anti-MIP1b, anti-IL4, anti-IL5, anti-IL17A, anti-IL6, anti-MCP1, anti-CD112, anti-CDD115, anti-CD34, anti-CD11b, anti-CD11a, anti-CD141, anti-CD105, anti-CD1a, anti-CD1c, anti-CD235a, anti-YTHDF2, anti-CD41a, anti-CD71, anti-Siglec7, anti-CD30, anti-CD61, anti-RANTES, anti-MCT-1, anti-MCT4, anti-CD80, anti-CD83, anti-CD86, anti-KIR3DL1, anti-KIR3DL2, anti-KIR2DL1, anti-NKG2C, anti-CD94, anti-NRF2, anti-CDT1, anti-pRB, anti-CyclinB1, anti-BrU and combinations thereof.

In some aspects, the metal-tagged probes are capable of detecting a cell target at a level of increased sensitivity compared to a metal-tagged probe that does not comprise a metal tag as described herein.

Further provided are methods of making the metal tags and labeled biomolecules described herein.

In some aspects, a method of generating a metal tag comprises providing an isotope salt; dissolving the isotope salt in a solvent; providing a polymer; and loading the isotope onto the polymer to generate a metal tag.

In some aspects, the isotope salt is selected from the group consisting of palladium (II) nitrate, cadmium (II) nitrate, indium (III) chloride hydrate, yttrium (III) chloride hydrate, yttrium nitrate hydrate, rhodium (III) chloride hydrate, ruthenium (III) chloride hydrate, lanthanum (III) nitrate hydrate, tin (II) chloride hydrate, mercury (II) nitrate hydrate, lead (II) nitrate, and rhenium (III) chloride.

In some aspects, the polymer used in the method is X8 or MCP9.

In some aspects, the solvent used in the method is selected from water, hydrochloric acid, nitric acid, 4-(2-hydroxyethyl)-1-piperazineethanesulfonic acid, and combinations thereof.

In some aspects, nitric acid is used as a solvent at a concentration between about 3% and about 5%.

In some aspects, the isotope salt is yttrium (III) chloride hydrate and the solvent is water.

In some aspects, the isotope salt is yttrium (III) chloride hydrate, the polymer is MCP9, and the solvent is water.

In some aspects, the isotope salt is yttrium (III) nitrate hydrate and the solvent is water.

In some aspects, the isotope salt is yttrium (III) nitrate hydrate, the polymer is MCP9, and the solvent is water or L buffer from Fluidigm®.

In some aspects, the isotope salt is ruthenium (III) chloride hydrate and the solvent is between about 3% nitric acid and about 5% nitric acid.

In some aspects, the isotope salt used in the method is ruthenium (III) chloride hydrate, the polymer is X8, and the solvent is about 3% nitric acid.

In some aspects, the ruthenium (III) chloride is loaded onto the X8 polymer in the presence of about 3% nitric acid.

In some aspects, the isotope salt is rhodium (III) chloride hydrate, and the solvent is water.

In some aspects, the isotope salt is rhodium (III) chloride hydrate, the polymer is MCP9, and the solvent is water (pH about 6) or L buffer from Fluidigm®.

In some aspects, the isotope salt is palladium (II) nitrate hydrate and the solvent is water.

In some aspects, the isotope salt is palladium (II) nitrate hydrate, the polymer is MCP9, and the solvent is water (pH about 6) or L buffer from Fluidigm®.

In some aspects, the isotope salt is cadmium (II) nitrate and the solvent is L buffer from Fluidigm®.

In some aspects, the isotope salt is cadmium (II) nitrate, the polymer is MCP9, and the solvent is L buffer from Fluidigm®.

In some aspects, the isotope salt is tin (II) chloride hydrate and the solvent is between about 3% nitric acid and about 5% nitric acid.

In some aspects, the isotope salt used in the method is tin (II) chloride hydrate, the polymer is MCP9, and the solvent is about 3% nitric acid.

In some aspects, the isotope salt is indium (III) chloride and the solvent is water (pH about 6) or L buffer from Fluidigm®.

In some aspects, the isotope salt is indium (III) chloride, the polymer is X8, and the solvent is water (pH about 6) or L buffer from Fluidigm®.

In some aspects, the isotope salt is mercury (II) nitrate hydrate and the solvent is about 5% nitric acid.

In some aspects, the isotope salt is mercury (II) nitrate hydrate, the polymer is MCP9, and the solvent is about 3% nitric acid.

In some aspects, the isotope salt is lead (II) nitrate and the solvent is water (pH about 6).

In some aspects, the isotope salt is lead (II) nitrate, the polymer is MCP9, and the solvent is water (pH about 6) or L buffer from Fluidigm®.

In some aspects, the isotope salt is rhenium (III) chloride and the solvent is between about 3% nitric acid and about 5% nitric acid.

In some aspects, the isotope salt is rhenium (III) chloride, the polymer is X8, and the solvent is about 3% nitric acid.

In some aspects, the isotope salt is lanthanum (III) nitrate hydrate, and the solvent is water (pH about 6).

In some aspects, the isotope salt is lanthanum (III) nitrate hydrate, the polymer is MCP9, and the solvent is water (pH about 6) or L buffer from Fluidigm®.

Further provided are methods of producing metal-tagged antibodies. In some aspects, the methods comprise providing an antibody; partially reducing the antibody to generate sulfhydryl groups; providing a metal tag that is generated according to the methods described herein; and conjugating the metal tag to the partially reduced sulfhydryl group of the antibody to produce the metal-tagged antibody.

Also provided are kits, panels, and system comprising the metal tags described herein.

In some aspects, the kits comprise a population of metal tags as described herein and one or more reagents that facilitate conjugation of at least one antibody to a metal tag of the population of metal tags described herein.

In some aspects, the kits comprise a population of metal-tagged probes as described herein.

In some aspects, the kits comprise antibodies, wherein each antibody targets a different antigen and all antibodies comprise the same metal tag.

In some aspects, the kits comprise antibodies, wherein each antibody targets a different antigen and some antibodies comprise the same metal tag and some antibodies comprise metal tags that are different from each other.

In some aspects, the kits comprise a plurality of antibodies and a plurality of reagents that comprise combinations of metal tags such that each antibody can be tagged with all combinations of metal tags.

In some aspects, the kits comprise a plurality of antibodies and a plurality of reagents for labeling the plurality of antibodies to generate a plurality of barcoding reagents to interrogate a plurality of samples, wherein each of the plurality of barcoding reagents when generated comprises a distinct combination of metal tags and/or antibodies each tagged with a distinct combination of metal tags.

In some aspects, the kits further comprise a calibration material comprising a known quantity of an isotope present in a metal tag of the population of metal tags and/or the population of metal-tagged probes described herein.

In some aspects, the kits further comprise a plurality of hermetically sealed containers.

In some aspects, each of the plurality of sample barcoding reagents of the kits is contained within different containers of the plurality of hermetically sealed containers.

Also provided are panels for elemental analysis comprising a plurality of conjugated antibodies, wherein each of the plurality of conjugated antibodies is tagged with a distinct metal tag as described herein, wherein each distinct metal tag is distinguishable based on its isotopic composition, and wherein the plurality of conjugated antibodies is in a mixture.

In some aspects, the plurality of conjugated antibodies of a panel include two or more antibodies selected from anti-CD45, anti-CD19, anti-CD3, anti-CD4, anti-CD8, anti-TNFα, anti-IFN-γ, anti-CD16, anti-CD33, anti-CD36, anti-phosphoSTAT5, anti-IL2, anti-CD28, anti-CD14, anti-T-bet, anti-CD66b, anti-B2M, anti-CD20, anti-HLA-DR, anti-CD14, anti-CD56, anti-CD294, anti-CD38, anti-CD11c, anti-CD57, anti-CD123, anti-TCRγδ, anti-CD161, anti-CCR7, anti-CD45RA, anti-CD27, anti-CD45RO, anti-CD25, anti-CCR5, anti-CCR4, anti-CXCR3, and anti-CCR6, anti-CD26, anti-KG2A, anti-CD49F, anti-CD127, anti-CCR2, anti-TIGIT, anti-Ki-67, anti-TIM3, anti-2B4, anti-ICOS, anti-PD-1, anti-Eomes, anti-CD137, anti-CTLA-4, anti-LAG3, anti-Perforin, anti-CD160, anti-CD69, anti-CD39, anti-KLRG1, anti-CD95, anti-GITR, anti-CLA, anti-CD73, Anti-CD298, anti-CD98, anti-G6PD, anti-GAPDH, anti-CD15S, anti-pS6, anti-pAKT, anti-MEK1/2, anti-pERK1/2, anti-phosphoSTAT3, anti-phosphoSTAT1, anti-phosphoSTAT4, anti-phosphoSTAT6, anti-p4EBP1, anti-GLUT1, anti-H3K27, anti-c-myc, anti-granzyme A, anti-granzyme B, anti-granzyme K, anti-granzyme M, anti-Helios, anti-RUNX3, anti-p53, anti-HIF1a, anti-p-NRF2, anti-Bcl-2, anti-Mcl-1, anti-Bcl-xL, anti-BIM, anti-Bax, anti-CD90, anti-CD96, anti-OX40, anti-MIP1a, anti-MIP1b, anti-IL4, anti-IL5, anti-IL17A, anti-IL6, anti-MCP1, anti-CD112, anti-CDD115, anti-CD34, anti-CD11b, anti-CD11a, anti-CD141, anti-CD105, anti-CD1a, anti-CD1c, anti-CD235a, anti-YTHDF2, anti-CD41a, anti-CD71, anti-Siglec7, anti-CD30, anti-CD61, anti-RANTES, anti-MCT-1, anti-MCT4, anti-CD80, anti-CD83, anti-CD86, anti-KIR3DL1, anti-KIR3DL2, anti-KIR2DL1, anti-NKG2C, anti-CD94, anti-NRF2, anti-CDT1, anti-pRB, anti-CyclinB1, anti-BrU, and any combination thereof.

In some aspects, at least two conjugated antibodies of the plurality of conjugated antibodies of the panel are tagged with distinct metal tags having different isotopes of a single element described herein.

In some aspects, the panel is contained in kits.

In some aspects, the kit comprising the panel further comprises a calibration material comprising a known quantity of an isotope present in a metal tag of an antibody of the plurality of conjugated antibodies.

Further provided are barcoding systems comprising: a plurality of barcoding reagents, wherein each of the plurality of barcoding reagents comprises one of a plurality of combinations of metal tags and/or metal-tagged probes as described herein.

In some aspects, each of the plurality of barcoding reagents of the barcoding system is contained within a container.

In some aspects, the container of the barcoding system is a hermetically sealed container.

In some aspects, the barcoding reagent of the barcoding system is a bead.

In some aspects, each metal tag of the combination of metal tags of the barcoding system is distinguishable through elemental analysis.

In some aspects, the barcoding system is contained in a barcoding kit.

In some aspects, the barcoding kit further comprises combinations of metal tags and/or metal tag probes to bind target analytes in a sample.

In some aspects, the target analytes are present on a cell.

In some aspects, the cell is a live cell.

Further provided are methods of using the metal tags and metal-tagged probes described herein.

In some aspects, provided is a method of interrogating or analyzing a sample comprising a cell with an elemental analyzer comprising mixing the sample with the population of metal-tagged probes as described herein to detect the presence of a metal tag on the cell.

In some aspects, the elemental analyzer used in the method is a mass cytometer and the method comprises performing mass cytometry.

In some aspects, the method comprises labeling a sample with a population of metal-tagged probes, wherein the population of metal-tagged probes bind to a target on a cell and barcode the cell; and detecting the barcoded cell based on the presence of the metal-tagged probe bound to the cell.

In some aspects, the method further comprises labeling a sample with a population of metal-tagged probes, wherein the population of metal-tagged probes bind to at least one analyte on a cell that is different from the molecule bound by the metal-tagged probe that barcoded the cell and detecting the at least one analyte on the barcoded cell.

In some aspects, the cell used in the method is a live cell.

In some aspects, the analyte on the cell is detected at a level of increased sensitivity compared to a metal-tagged probe that does not comprise a metal tag as described herein.

In some aspects, the methods described herein are used to stain cells in a tissue section sample.

### BRIEF DESCRIPTION OF THE DRAWINGS/FIGURES

FIG. 1A shows histograms of signal intensities for 7 Cd isotopes tagged to CD45 antibody and antibody capture beads (bottom row) compared to background signal (top row). To estimate background signal intensities, a Cd isotope other than the Cd isotope of interest was used. FIG. 1B shows signal intensities for Pd and Cd isotopes with overlapping mass weights. Top row histograms show background signal intensities of isotopes having mass weights of 106 and 110. FIG. 1C shows biaxial plots of signal intensities for serial dilution of CD45 tagged to 110Cd and 116Cd isotopes using PBMCs.

FIG. 1D shows a spill-over matrix for 3 Pd and 7 Cd isotopes. The numbers in the cells represent the percentage of spill-over.

FIG. 2 shows biaxial plots of signal intensities for serial dilutions of CD45 antibodies tagged with 5 different Cd isotopes, 106Cd, 111Cd, 112Cd, 113Cd and 114Cd.

FIG. 3A shows histograms of signal intensities for 6 Pd isotopes, 102Pd (red), 104Pd, 105Pd 106Pd 108Pd and 110Pd on PBMCs labeled with MCP9-loaded natural abundance Pd. FIG. 3B shows biaxial plots of signal intensities of PBMCs from a healthy donor stained separately with 10 different MCBs (mass-tag cell barcodes), pooled and acquired simultaneously. FIG. 3C shows Umap plots implemented with a FlowSOM algorithm using expression levels for the 10 different tags. Umap plots were grayscaled based on arcsin h values for each parameter utilized for dimension reduction and clustering.

FIG. 4A shows histograms of signal intensities for three MCP9-loaded Pd isotopes, 104Pd, 105Pd and 108Pd, tagged with CD45 compared to background signal intensities. FIG. 4B shows a bubble plot of mean signal intensities and staining indices for 104Pd, 105Pd, and 108Pd. FIG. 4C shows histograms of signal intensities for Cd and Pd isotopes loaded onto MCP9 or mDOTA. FIG. 4D shows signal intensities for the 10 MCBs in biaxial plots. PBMCs from a healthy donor were separately labeled with the 10 different MCBs utilizing MCP9 polymers loaded with 7 Cd and 3 Pd isotopes. Samples were then pooled and acquired simultaneously. FIG. 4E shows pooled samples (n=10) subjected to dimension reduction and clustering using UMAP and FlowSOM algorithms, respectively. Each grayscale code corresponds to a single sample.

FIG. 5A shows staining indices for 5 Cd-tagged antibodies used alone or in combinations of 2 and 3 antibodies. FIG. 5B shows two-dimensional t-SNE maps of the samples of FIG. 5D grayscaled for 10 Cd or Pd-tagged CD45 antibodies. FIG. 5C shows pooled samples deconvoluted using Premessa R package and de-barcoded shown per assigned barcoding tags (n=45). FIG. 5D shows a purity matrix of fractional abundance of samples in each deconvoluted sample.

FIG. 6A shows a schematic representation of a barcoding scheme (created with BioRender.com). FIG. 6B shows biaxial plots of signal intensities of 45 unique dual combinations generated from a pool of 10 Pd and Cd tagged CD45 antibodies using a 10-choose-2 scheme to barcode 45 different experimental conditions. FIG. 6C shows histograms of signal intensities of Pd or Cd tagged CD45 antibodies when Cd or Pd-tagged CD45 antibodies were used alone or in dual combinations with either Cd or Pd-tagged CD45 antibodies to label PBMCs. FIG. 6D shows a heatmap of the mean signal intensities for the 10 MCBs in FIG. 6C. Values are scaled per row. FIG. 6E shows a two-dimensional t-SNE plot of the pooled 45 samples in FIG. 6B on a high-dimensional plane. FIG. 6F shows a heatmap of the arcsin h-transformed mean signal intensities of CD45 antibodies tagged to 10 different isotopes used to barcode 45 samples in FIG. 6E. FIG. 6G shows 45 pooled samples deconvoluted using Premessa R package. Graph on the upper right shows values of about 2.5-3 for isotopes 104Pd and 105Pd and values of less than 1 for isotopes 106Cd, 108Pd, 110Cd, 111Cd, 112Cd, 113Cd, 114Cd, and 116Cd.

FIG. 7A shows histograms of signal intensities of CD298, CD45, and B2M on PBMCs incubated with CD298, CD45, and B2M antibodies tagged with 114Cd compared to background signal intensity. FIG. 7B shows t-SNE maps of the data in FIG. 7C grayscaled based on expression of 5 CD45-based tags used for sample barcoding. Scale shows arcsin h values. FIG. 7C shows deconvoluted samples from two healthy donors (HD) (left, HD1 and right HD2) plotted per assigned barcoding tags. FIG. 7D shows PhenoGraph clusters observed in deconvoluted samples from the 2 HDs in FIG. 7C. FIG. 7E shows a heatmap of marker expression levels for 23 PhenoGraph clusters in FIG. 7D. Arcsin h values are shown.

FIG. 8A shows two-dimensional t-SNE maps of pooled samples of PBMCs from two HDs labeled with 106Cd_B2M and 108Pd_B2M antibodies separately and pooled. FIG. 8B shows a heatmap of scaled values for clusters shown in FIG. 8A. FIG. 8C shows contour t-SNE plots for an equal number of cells showing an immune landscape of HD1 (106Cd_B2M) and HD2 (108Pd_B2M). FIG. 8D shows cluster frequencies identified across two HDs using the PhenoGraph algorithm. FIG. 8E shows PhenoGraph clusters of deconvoluted samples (n=20) from two HDs plotted on a t-SNE analysis. Each dot represents a single sample. FIG. 8F shows a heatmap showing a correlation among deconvoluted samples (FIG. 8E) based on PhenoGraph cluster frequencies in FIG. 8D.

FIG. 9A shows histograms of 89Y signal intensities of two CD45 antibodies tagged with either MCP9 or X8 polymer loaded with 89Y. FIG. 9B shows cumulative results for multiple measurements.

FIG. 10A shows a histogram of the signal intensity for 103Rh. FIG. 10B shows cumulative results for multiple measurements.

FIG. 11A shows antibody capture beads labeled with CD45 antibody tagged with X8 polymer loaded with Ruthenium (Ru) isotopes. Signal intensities varied across different isotopes and reflect the natural abundance of isotopes in RuCl₃·xH₂O used for antibody conjugation. 97Molybdenum (97Mo) was used as a negative control. FIG. 11B shows the OCI-AML3 cell line labeled with CD45 antibody tagged with X8 polymer loaded with Ru isotopes.

FIG. 12A shows signal intensities for 10 Sn isotopes. Tin isotopes were loaded onto MCP9 polymer and tagged to CD45 antibody. Capture beads were used for signal quantification. FIG. 12B shows signal intensities of the OCI-AML3 cell line labeled with CD45 antibody tagged with MCP9 polymer loaded with 7 Sn isotopes. Signal intensities for 111Cd is shown as a measure of background signal intensities.

FIG. 13A shows signal intensities for 139La for three antibody conjugates. 139La-X8 conjugates were not stable while 139La-MCP9 conjugates tagged to antibodies were stable and elicited high signal intensities. CD36, CD45 and CD8 antibodies were conjugated with MCP9 polymer loaded with 139La. FIG. 13B shows 139La signal intensity of OCI-AML3 cells and peripheral blood mononuclear cells from a healthy donor mixed and stained with 139La-CD36 antibody. FIG. 13C shows signal intensities of 140Ce (cerium) captured by X8 and MCP9 polymers and tagged to CD8 antibody.

FIG. 14A shows signal intensities for 7 Hg isotopes loaded on MCP9 and tagged to antibodies. Capture beads were labeled with metal antibodies of interest and were used for signal quantification. FIG. 14B shows signal intensities of the OCI-AML3 cell line labeled with CD45 antibody tagged with MCP9 polymer loaded with natural abundance Mercury salt and signal intensities for 7 Hg isotopes are shown. Signal intensities for 192Os is shown as a measure of background signal intensities.

FIG. 15A shows signal intensities for 4 Pb isotopes loaded on MCP9 and tagged to antibodies on capture beads. Lead-MCP9 conjugate was tagged to CD45 and CD8 antibodies. Antibody capture beads were stained with 0.5 ug of Lead-CD8 antibody conjugate. FIG. 15B shows signal intensities of the OCI-AML3 cell line labeled with CD45 antibody tagged with MCP9 polymer loaded with 4 Pb isotopes.

### DETAILED DESCRIPTION

The present disclosure provides compositions of isotopes and polymers useful for elemental analysis using mass cytometry (CyTOF). The compositions comprise populations of metal tags, wherein each metal tag comprises a polymer and an isotope.

In some aspects, the polymer comprises at least one metal-chelating group. In some aspects, the polymer comprises between one and about 1000 metal-chelating groups.

In some aspects, the polymer is selected from X8 polymer and metal-chelating polymer 9 (MCP9).

In some aspects, the isotope has an atomic mass between about 89 and about 208.

In some aspects, the isotope is selected from cadmium, palladium, indium, yttrium, and combinations thereof.

In some aspects, a population of metal tags provided herein comprises at least one isotope that is 103Rh. In some aspects, the population of metal tags further comprises one or more isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In. In some aspects, a population of metal tags comprises an isotope that is selected from yttrium, ruthenium, rhodium, tin, lanthanum, mercury, lead, and rhenium.

In some aspects, the population of metal tags comprises at least one isotope that is selected from 185Re and 187Re. In some aspects, the population of metal tags further comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

In some aspects, a population of metal tags comprises an isotope selected from 196Hg, 198Hg, 199Hg, 200Hg, 201Hg, 202Hg, and 204Hg. In some aspects, the population of metal tags further comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re and 187Re.

In some aspects, a population of metal tags comprises an isotope selected from 204Pb, 206Pb, 207Pb, and 208Pb. In some aspects, the population of metal tags further comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, a population of metal tags comprises a 139La isotope.

In some aspects, the population of metal tags further comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re and 187Re.

In some aspects, provided is a population of metal tags that comprises at least one metal tag that comprises one or more isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In and at least one metal tag that comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, provided is a population of metal tags that comprises at least one metal tag that comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, and 208Pb and at least one metal tag that comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re and 187Re.

In some aspects, the population of metal tags comprises a polymer that is X8 polymer and one or more isotopes selected from 96Ru, 87Ru, 99Ru, 100Ru, 101Ru, 102Ru, and 104Ru.

In some aspects, the population of metal tags comprises isotopes 103Rh, 89Y, and 139La.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by those skilled in the art. With regard to the definitions and terms in the art, reference may be made by those skilled in the art to, for example, Mass Cytometry Method and Protocols (H M McGuire & T M Ashhurst).

The term “about,” as used herein, refers to a value or composition that is within an acceptable error range for the particular value or composition as determined by one of ordinary skill in the art, which will depend in part on how the value or composition is measured or determined, i.e., the limitations of the measurement system. For example, about can include the recited number±10% (for example, “about 10” means 9 to 11).

Although the numerical ranges and approximate parameter values are shown in a broad range in the present invention, all the numerical values set forth in the specific examples are described as precisely as possible. However, any numerical value inherently contains certain errors necessarily resulting from the standard deviation found in their respective measurements. Additionally, all ranges disclosed herein are to be understood to encompass any and all subranges subsumed therein. For example, a stated range “1 to 10” should be considered to include any and all subranges between (and inclusive of) the minimum value of 1 and the maximum value of 10; that is, all subranges beginning with a minimum value of 1 or more, such as 1 to 6.1, and ending with a maximum value of 10 or less, such as 5.5 to 10. Additionally, any reference referred to as being “incorporated herein” is to be understood as being incorporated in its entirety.

The singular forms “a,” “an,” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “metal” as used herein refers to an element having one of the following atomic numbers 3, 4, 11-13, 19-33, 37-52, 55-84, 87-102.

The term “transition metal” as used herein refers to an element having one of the following atomic numbers 21-30, 39-48, 57-80 and 89-108. Transition elements include the rare earth metals, lanthanides and noble metals.

The term “lanthanides” as used herein refers to transition metals with atomic numbers from 57 to 71 including La, Ce, Pr, Nd, Pm, Sm, Eu, Gd, Tb, Dy, Ho, Er, Tm, Yb, Lu.

The term “isotopes” as used herein refers to two or more types of atoms that have the same atomic number (number of protons in their nucleus) and position in the periodic table (and, thus, belong to the same chemical element) and that differ in nucleon numbers (mass numbers) due to different numbers of neutrons in their nuclei. The isotopes as referred to herein include isotopes of an atomic mass between about 89 and about 208.

The term “tag atom” or “metal atom” is the atom of the element or isotope that differentiates one element tag or metal tag from another and that is detected by elemental analysis.

The term “metal tag” or “tag” or “element tag” as used herein refers to a chemical moiety which includes an element or multitude of elements having one or many isotopes (referred to as “tag atoms” or “metal atoms”) attached to a supporting molecular structure, or a structure that is capable of binding said element(s) or isotope(s). The element tag or metal tag can also comprise the means of attaching the element tag or metal tag to a molecule of interest or target molecule (for example, an analyte). Different element tags or metal tags may be distinguished on the basis of the elemental composition of the tags. An element tag or metal tag can contain many copies of a given isotope and can have a reproducible copy number of each isotope in each tag. An element tag or metal tag is functionally distinguishable from a multitude of other element tags or metal tags in the same sample because its elemental or isotopic composition is different from that of the other tags.

The term “polymer” as used herein refers to a substance composed of molecules characterized by multiple repetitions of one or more species of atoms or groups of atoms (constitutional units) linked to each other in amounts sufficient to provide a set of properties that do not vary markedly with the addition or removal of one or a few constitutional units. A polymer molecule can be characterized in terms of its backbone, the connected link of atoms that span the length of the molecule, and the pendant groups, attached to the backbone portion of each constituent unit. The pendant groups are often chemically and functionally different from the backbone chain. Pendant groups that have a high affinity for metal ions can act as chelating groups or ligands for those ions. A polymer includes “copolymers” that are polymers that consist of two or more chemically different constituent units. A polymer includes a “linear polymer” that is a polymer characterized by a linear sequence of constituent units. A polymer includes a “block copolymer” that is a linear polymer with sequences of constituent units of a common type joined to sequences of constituent units of a different type. A polymer includes a “branched polymer” that is a polymer in which additional polymer chains or branches originate from the backbone of the polymer. The longest linear sequence is referred to as the “main chain.” A branched polymer in which the chemical composition of the constituent units of the branch chains is different than those of the main chain is called a “graft copolymer.” A polymer includes a “star polymer” that has multiple linear polymer chains emanating from a common constituent unit or core. A polymer includes a “hyperbranched polymer” that comprises multiple branched polymers in which the backbone atoms are arranged in the shape of a tree. These polymers are related to “dendrimers”, which have three distinguishing architectural features: an initiator core, interior layers (generations) composed of repeating units radially attached to the initiator core, and an exterior surface of terminal functionality attached to the outermost generation. “Dendrimers” differ from hyperbranched polymers by their extraordinary symmetry, high branching, and maximized terminal functionality.

The term “metal tagged polymer” (also a “metal-polymer conjugate”) as used herein refers to a variety of the metal tag which consists of a polymer backbone bearing at least one chelating group with metal atoms attached to them. These metal tagged polymers can be, but are not limited to, linear, star, branched, or hyperbranched homopolymers or copolymers as well as block or graft copolymers.

The term “X8 polymer” as used herein refers to Fluidigm®'s straight-chain X8 polymer.

The term “MCP9 polymer” as used herein refers to Fluidigm®'s MCP9 polymer.

The term “metal binding group” as used herein refers to a group on the polymer that is capable of binding a metal or an isotope of a metal. It can also be referred to as a ligand.

The term “chelation” as used herein refers to a process of binding of a ligand, the chelant, chelator or chelating agent, to a metal ion, forming a metal complex, the chelate. In contrast to the simple monodentate ligands like H₂O or NH₃, the polydentate chelators form multiple bonds with the metal ion.

The term “population of metal tags” as used herein refers to a plurality of metal tags, wherein each metal tag can be distinguishable from the other metal tags of the population or more than one metal tag can comprise a polymer and/or metal or isotope that is similar to one or more of the other metal tags of the population of metal tags.

The term “metal-tagged probe” as used herein refers to a metal tag that further comprises a binding reagent attached to the metal tag either covalently or non-covalently.

The term “binding reagent” as used herein refers to a molecule that is capable of binding to a target molecule or analyte, for example an antigen or a biomarker and is conjugated to a metal tag. A binding agent can be a biomolecule. In some aspects, the binding reagent is conjugated to a metal tag through a linker group. For example, streptavidin is a binding reagent that specifically binds biotin. In some aspects, the binding reagent is an antibody and is conjugated to the metal tag via a sulfhydryl group.

The term “biomolecule” as used herein refers to any biological molecule and includes, but is not limited to, small biomolecules including lipids, phospholipids, glycolipids, sterols, vitamins, hormones, growth factors, neurotransmitters, carbohydrates, sugars, disaccharides, amino acids, nucleotides, phosphate, and monosaccharides; and large biomolecules including, but not limited to, peptides, oligopeptides, polypeptides, proteins, nucleic acids, i.e. DNA, RNA, oligonucleotides, aptamers, oligosaccharides, polysaccharide, lectins, and prions. Other biomolecules are known to those skilled in the art and are encompassed in the disclosure.

The term “barcoding system” as used herein refers to a plurality of barcoding reagents comprising a plurality of metal tag barcodes.

The term “n-choose-2 barcoding scheme” refers to a barcoding approach where (n*(n−1))/2 experimental conditions can be barcoded; “n” denotes the number of different metal tags used and determines the barcoding depth. For example, a 6-choose-2 scheme can be used to barcode 15 different experimental conditions.

The term “lower end of the sensitivity spectrum” as used herein refers to an isotope of a mass below about 139, where the mass of an isotope is correlated with the sensitivity of its detection by mass cytometry. The term “higher end of the sensitivity spectrum” as used herein refers to an isotope of a mass between about 153 and about 176. The term “intermediate sensitivity” refers to an isotope of a mass between about 139 and about 152.

The term “solvent” as used herein refers to any substance that dissolves any of the solutes described herein.

The term “panel” as used herein refers to a plurality of reagents comprising a plurality of mass cell tag barcodes and, optionally, a support entity. In some aspects, the support entity is a solid support. In some aspects, the support entity is a bead.

The term “calibration material” as used herein refers to a material comprising a known quantity of an isotope. In some aspects, the calibration material is included in a kit as described herein and comprises a known quantity of at least one isotope that is present in at least one metal tag as described herein.

The term “hermetically sealed container” as used herein refers to any container made of any material that encloses its content airtight, that is prevents the passage of air, oxygen, or other gases.

The term “bead” as used herein refers to a polymer particle with a diameter between about 500 nm and about 500 μm that has a uniform shape, can absorb or be coupled to bioreactive molecules and can be used to separate proteins, nucleic acids, and/or cells.

The terms “elemental analysis” and “elemental analyzer” as used herein refers to a method or machine useful for analyzing the presence of an element or isotope in a sample and includes a mass cytometer.

The term “analyte” as used herein refers to any substance whose chemical constituents or functional characteristics are being measured or identified.

The term “live cell” as used herein refers to any cell that is alive and has not been fixed or preserved prior to being analyzed by a method described herein.

The term “antibody” as used herein means a proteinaceous molecule belonging to the immunoglobulin class of proteins, containing one or more domains that bind an epitope on an antigen, where such domains are or are derived from or share sequence homology with the variable region of an antibody. Antibodies are typically made of basic structural units—each with two heavy chains and two light chains. An antibody described herein is not limited to any particular format or method of producing it. As used herein, antibody encompasses full length antibodies (e.g., IgG1 or IgG4 antibodies), various functional fragments thereof (e.g., those which may comprise only antigen-binding moieties, such as Fab, F(ab′)2 or scFv fragments), as well as modified antibodies (e.g., humanized, glycosylated etc.).

### II. Metal Tags

In some aspects, provided is a metal tag comprising a polymer and at least one metal atom or isotope. In some aspects, the polymer comprises at least one metal-binding group that comprises at least one metal atom or isotope or is capable of binding at least one metal atom or isotope. The metal tag can further comprise a functional group that allows the polymer to be attached to one of a linker, a spacer, or a biomolecule. The metal tag can be water soluble.

In some aspects, the metal tag comprises a polymer that comprises metal-binding groups capable of binding at least one metal atom or isotopes or between about 1 and 1000 metal atoms or isotopes, between about 2 and about 800, about 5 and about 750, about 10 and about 600, about 50 and about 550, about 75 and about 500, about 100 and 400 or about 1, 2, 3, 4, 5, 6, 7 8, 9, 10, 11, 12, 13, 14, 15, 16, 17, 18, 19, 20 21, 22, 23, 24, 25, 26, 27, 28, 29, 30, 31, 32, 33, 34, 35, 36 37, 38, 39, 40, 41, 42, 43, 44, 45, 46 47, 48, 49 and 50; about 10 to about 50, about 51 to about 75, about 76 to about 100, about 101 to about 125, about 126 to about 150, about 151 to about 175, about 176 to about 200, about 201 to about 250, about 251 to about 300, about 301 to about 350, about 351 to about 400, about 401 to about 450, about 451 to about 500, about 501 to about 550, about 551 to about 600, about 601 to about 650, about 651 to about 700, about 701 to about 750, about 751 to about 800, about 801 to about 850, about 851 to about 900, about 901 to about 950, or about 951 to about 1000 metal atoms or isotopes.

In some aspects, the number of metal-binding groups capable of binding at least one metal atom or isotope is between about 10 and about 250. In some aspects, at least one metal atom or isotope can be bound to at least one of the metal-binding groups.

In some aspects, the polymer can have a degree of polymerization of between about 1 and about 1000, or between about 2 and about 800, about 5 and about 750, about 10 and about 600, about 50 and about 550, about 75 and about 500, about 100 and 400; or about 10 to about 50, about 51 to about 75, about 76 to about 100, about 101 to about 125, about 126 to about 150, about 151 to about 175, about 176 to about 200, about 201 to about 250, about 251 to bout 300, about 301 to about 350, about 351 to about 400, about 401 to about 450, about 451 to about 500, about 501 to about 550, about 551 to about 600, about 601 to about 650, about 651 to about 700, about 701 to about 750, about 751 to about 800, about 801 to about 850, about 851 to about 900, about 901 to about 950, or about 951 to about 1000.

In some aspects, the polymer is selected from the group consisting of linear polymers, co-polymers, branched polymers, graft copolymers, block polymers, star polymers, and hyperbranched polymers. In some aspects, the backbone of the polymer is derived from substituted polyacrylamide, polymethacrylate, or polymethacrylamide and can be a substituted derivative of a homopolymer or copolymer of acrylamides, methacrylamides, acrylate esters, methacrylate esters, acrylic acid or methacrylic acid.

In some aspects, the metal-binding group is attached to the polymer through an ester or through an amide. In some aspects, the polymer comprises a functional group that is a thiol-reactive group.

In some aspects, the metal atom or isotope is a transition metal.

In some aspects, the metal atom or isotope is selected from one or more of cadmium, palladium, indium, yttrium, and combinations thereof.

In some aspects, the metal atom or isotope is selected from one or more of yttrium, ruthenium, rhodium, tin, lanthanum, lead, and combinations thereof.

In some aspects, the metal atom or isotope is selected from one or more yttrium, ruthenium, rhodium, tin, lanthanum, mercury, rhenium, and combinations thereof.

In some aspects, the metal atom or isotope is selected from one or more of 106Cd, 110Cd, 111Cd, 112Cd, 113Cd, 114Cd, 116Cd, 102Pd, 104Pd, 105Pd, 106Pd, 108Pd, 110Pd, 89Y, and 115In.

In some aspects, the metal atom or isotope is selected from one or more of 106Cd, 110Cd, 111Cd, 112Cd, 113Cd, 114Cd, 116Cd, 102Pd, 104Pd, 105Pd, 108Pd, 89Y, and 115In.

In some aspects, the metal atom or isotope is selected from one or more of 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, and 208Pb.

In some aspects, the metal atom or isotope is selected from one or more of 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

In some aspects, the metal atom or isotope is selected from one or more of 106Cd, 110Cd, 111Cd, 112Cd, 113Cd, 114Cd, 116Cd, 102Pd, 104Pd, 105Pd, 106Pd, 108Pd, 110Pd, 89Y, and 115In and one or more of 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, and 208Pb.

In some aspects, the metal atom or isotope is selected from one or more of 106Cd, 110Cd, 111Cd, 112Cd, 113Cd, 114Cd, 116Cd, 102Pd, 104Pd, 105Pd, 106Pd, 108Pd, 110Pd, 89Y, and 115In and one or more of 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

In some aspects, the metal atom or isotope is selected from one or more of 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, and 208Pb, and one or more of 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

In some aspects, the metal atom or isotope is selected from one or more of 106Cd, 110Cd, 111Cd, 112Cd, 113Cd, 114Cd, 116Cd, 102Pd, 104Pd, 105Pd, 106Pd, 108Pd, 110Pd, 89Y, and 115In, one or more of 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re, and one or more of 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, and 208Pb.

### III. Metal-Tagged Probes

In some aspects, the metal tags provided herein are conjugated to biomolecules to generate metal-tagged probes.

In some aspects, the metal-tagged probe comprises a metal tag as described herein and a binding reagent. In some aspects, the binding reagent is a polypeptide, protein, nucleic acid, and/or polysaccharide.

In some aspects, the metal tag can comprise a linker attached to the functional group of the polymer, wherein the linker is capable of covalent attachment to a binding reagent. In some aspects, the metal tag can further comprise a spacer attached to the linker, wherein the spacer is capable of attachment to a binding reagent.

In some aspects, the spacer is a polyethylene glycol (PEG) spacer. In some aspects, the spacer comprises a functional group that is capable of binding the spacer to the polymer via a spacer-reactive functional group on the polymer.

In some aspects, the metal tag is covalently attached to a binding reagent.

In some aspects, the binding reagent is an antibody. In some aspects, the binding reagent is a partially reduced antibody.

In some aspects, the antibody is tagged with the metal tag described herein, wherein at least one of the metal-binding groups of the polymer of the metal tag binds, or is capable of binding, at least one metal atom.

In some aspects, the polymer is X8 polymer or MCP9 polymer and the antibody is conjugated to the polymer through a sulfhydryl group.

In some aspects, the antibody is selected from an anti-CD45, anti-CD19, anti-CD3, anti-CD4, anti-CD8, anti-TNFα, anti-IFN-γ, anti-CD16, anti-CD33, anti-CD36, anti-phosphoSTAT5, anti-IL2, anti-CD28, anti-CD14, anti-T-bet, anti-CD66b, anti-B2M, anti-CD20, anti-HLA-DR, anti-CD14, anti-CD56, anti-CD294, anti-CD38, anti-CD11c, anti-CD57, anti-CD123, anti-TCRγδ, anti-CD161, anti-CCR7, anti-CD45RA, anti-CD27, anti-CD45RO, anti-CD25, anti-CCR5, anti-CCR4, anti-CXCR3, and anti-CCR6, anti-CD26, anti-KG2A, anti-CD49F, anti-CD127, anti-CCR2, anti-TIGIT, anti-Ki-67, anti-TIM3, anti-2B4, anti-ICOS, anti-PD-1, anti-Eomes, anti-CD137, anti-CTLA-4, anti-LAG3, anti-Perforin, anti-CD160, anti-CD69, anti-CD39, anti-KLRG1, anti-CD95, anti-GITR, anti-CLA, anti-CD73, Anti-CD298, anti-CD98, anti-G6PD, anti-GAPDH, anti-CD15S, anti-pS6, anti-pAKT, anti-MEK1/2, anti-pERK1/2, anti-phosphoSTAT3, anti-phosphoSTAT1, anti-phosphoSTAT4, anti-phosphoSTAT6, anti-p4EBP1, anti-GLUT1, anti-H3K27, anti-c-myc, anti-granzyme A, anti-granzyme B, anti-granzyme K, anti-granzyme M, anti-Helios, anti-RUNX3, anti-p53, anti-HIF1a, anti-p-NRF2, anti-Bcl-2, anti-Mcl-1, anti-Bcl-xL, anti-BIM, anti-Bax, anti-CD90, anti-CD96, anti-OX40, anti-MIP1a, anti-MIP1b, anti-IL4, anti-IL5, anti-IL17A, anti-IL6, anti-MCP1, anti-CD112, anti-CDD115, anti-CD34, anti-CD11b, anti-CD11a, anti-CD141, anti-CD105, anti-CD1a, anti-CD1c, anti-CD235a, anti-YTHDF2, anti-CD41a, anti-CD71, anti-Siglec7, anti-CD30, anti-CD61, anti-RANTES, anti-MCT-1, anti-MCT4, anti-CD80, anti-CD83, anti-CD86, anti-KIR3DL1, anti-KIR3DL2, anti-KIR2DL1, anti-NKG2C, anti-CD94, anti-NRF2, anti-CDT1, anti-pRB, anti-CyclinB1, anti-BrU, and any combination thereof.

In some aspects, the metal-tagged antibody probes are used for barcoding a sample, wherein a barcode identifies a sample in a pool of samples.

In some aspects, the metal-tagged probes are capable of detecting a cell target at a level of increased sensitivity compared to a metal-tagged probe that does not comprise a metal tag as described herein.

### IV. Methods of Preparing a Metal Tag

Provided are the methods for preparing a metal tag, wherein the methods comprise providing a polymer that comprises at least one metal-binding group capable of binding at least one metal atom and loading at least one metal atom to the at least one metal-binding group of the polymer.

In some aspects, the polymer used in the method is X8 or MCP9.

In some aspects, the methods comprise preparing a polymer suitable for binding at least one metal atom. In some aspects, the methods comprise: (i) providing a polymer; (ii) covalently attaching at least one metal-binding group containing at least one metal atom or capable of binding at least one metal atom to the polymer, and (iii) loading at least one metal atom to the metal-binding group of the polymer.

In some aspects, the method comprises functionalizing a polymer, wherein the functional group is capable of covalently binding a biomolecule.

In some aspects, the methods comprise binding or loading at least one metal atom to at least one metal-binding group. In some aspects, the metal atom binding or metal atom loading step is performed in water. In some aspects, the metal atom binding or metal atom loading step is performed in a buffer containing a solvent.

In some aspects, the method of generating a metal tag comprises providing an isotope salt; dissolving the isotope salt in a solvent; providing a polymer; and loading the isotope onto the polymer to generate a metal tag.

In some aspects, the isotope salt is selected from the group consisting of palladium (II) nitrate hydrate, cadmium (II) nitrate, indium (III) chloride, yttrium (III) chloride hydrate, yttrium (III) nitrate hydrate, rhodium (III) chloride hydrate, ruthenium (III) chloride hydrate, lanthanum (III) nitrate hydrate, tin (II) chloride hydrate, mercury (II) nitrate hydrate, lead (II) nitrate, and rhenium (II) chloride.

In some aspects, the isotope salt is dissolved in water. In some aspects, the isotope salt is dissolved in a solvent. In some aspects, the isotope salt is dissolved in water of a pH about 5.0 to about 7.0, In some aspects, the isotope salt is dissolved in water of a pH about 6. In some aspects, the isotope salt is dissolved in water of a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. In some aspects, the isotope salt is dissolved in a solvent of a pH about 5.0 to about 7.0, In some aspects, the isotope salt is dissolved in a solvent of a pH about 6. In some aspects, the isotope salt is dissolved in a solvent of a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0.

In some aspects, the solvent used in the method is selected from water, hydrochloric acid, nitric acid, 4-(2-hydroxyethyl)-1-piperazineethanesulfonic acid, and combinations thereof.

In some aspects, the solvent is hydrochloric acid and is used in a range of about 0.1% to about 8%; about 0.2% to about 7%; 0.4% to about 6%; about 0.6% to about 5%; about 0.8% to about 4%; or about 0.1% to about 3%. In some aspects, the hydrochloric acid is used at about 0.2%; about 0.5%, about 0.7%, about 1%, about 1.5%, about 2% about 2.5%, about 3%, about 3.5%, about 4%, about 4.5%, about 5%, about 5.5%, about 6%, about 6.5%, about 4%, about 7.5%, or about 8%.

In some aspects, the solvent is nitric acid and is used on a range of about 0.1% to about 8%; about 0.2% to about 7%; 0.4% to about 6%; about 0.6% to about 5%; about 0.8% to about 4%; or about 0.1% to about 3%. In some aspects, the hydrochloric acid is used at about 0.2%; about 0.5%, about 0.7%, about 1%, about 1.5%, about 2% about 2.5%, about 3%, about 3.5%, about 4%, about 4.5%, about 5%, about 5.5%, about 6%, about 6.5%, about 4%, about 7.5%, or about 8%.

In some aspects, the isotope salt is yttrium (III) chloride and the solvent is water (pH about 6). In some aspects, the yttrium (III) chloride is loaded onto MCP9 polymer. In some aspects, the isotope salt is yttrium (III) nitrate and the solvent is water (pH about 6). In some aspects, the yttrium (III) nitrate is loaded onto MCP9 polymer.

In some aspects, the isotope salt is ruthenium (III) chloride hydrate and the solvent is between about 3% nitric acid and about 5% nitric acid. In some aspects, the isotope salt is ruthenium (III) chloride hydrate and the solvent is about 3% nitric acid. In some aspects, the ruthenium (III) chloride hydrate is loaded onto the polymer in the presence of 3% nitric acid. In some aspects, the ruthenium (III) chloride hydrate is loaded onto X8 polymer. In some aspects, the ruthenium (III) chloride hydrate is loaded onto X8 polymer in the presence of 3% nitric acid.

In some aspects, the isotope salt is rhodium (III) chloride hydrate and the solvent is water (pH about 6). In some aspects, the rhodium (III) chloride is loaded onto MCP9 polymer.

In some aspects, the isotope salt is palladium (II) nitrate hydrate and the solvent is water (pH about 6). In some aspects, the palladium (II) nitrate is loaded onto MCP9 polymer.

In some aspects, the isotope salt is cadmium (II) nitrate and the solvent is L buffer from Fluidigm® (pH about 6). In some aspects, the cadmium (II) nitrate is loaded onto MCP9 polymer.

In some aspects, the isotope salt is tin (II) chloride hydrate and the solvent is between about 3% nitric acid and about 5% nitric acid. In some aspects, the isotope salt is tin (II) chloride hydrate and the solvent is about 5% nitric acid. In some aspects, the tin (II) chloride is loaded onto MCP9 polymer.

In some aspects, the isotope salt is indium (III) chloride and the solvent is water (pH about 6). In some aspects, the indium (III) chloride is loaded onto X8 polymer.

In some aspects, the isotope salt is mercury (II) nitrate hydrate and the solvent is between about 3% nitric acid and about 5% nitric acid. In some aspects, the isotope salt is mercury (II) nitrate hydrate and the solvent is about 5% nitric acid. In some aspects, the mercury (II) nitrate is loaded onto MCP9 polymer.

In some aspects, the isotope salt is lead (II) nitrate and the solvent is water (pH about 6). In some aspects, the lead (II) nitrate is loaded onto MCP9 polymer.

In some aspects, the isotope salt is rhenium (III) chloride and the solvent is between about 3% nitric acid and about 5% nitric acid. In some aspects, the isotope salt is rhenium (III) chloride and the solvent is about 3% nitric. In some aspects, the rhenium (III) chloride is loaded onto X8 polymer. In some aspects, the rhenium (III) chloride is loaded onto X8 polymer in the presence of water (pH about 6).

In some aspects, the isotope salt is lanthanum (III) nitrate hydrate and the solvent is water (pH about 6). In some aspects, the lanthanum (III) nitrate is loaded onto MCP9 polymer.

In some aspects, the polymer is X8 polymer and the X8 polymer is dissolved in about 3% nitric acid to about 5% nitric acid.

In some aspects, the polymer is X8 polymer and the X8 polymer is dissolved in about 3% nitric acid.

In some aspects, the polymer is polymer and the X8 polymer is dissolved in water (pH about 6).

In some aspects, the polymer is MCP9 polymer and the MCP9 polymer is dissolved in L buffer from Fluidigm® (pH about 6).

In some aspects, the metal tag is a yttrium tag, a ruthenium tag, a rhodium tag, a paladium tag, a cadmium tag, a tin tag, an indium tag, mercury tag, a lead tag, or lanthanum tag and is washed in a 3K filter comprising a polyethersulfone membrane.

In some aspects, the metal tag is a rhenium tag and is washed in a 3K filter comprising a low-binding regenerated cellulose membrane.

### V. Methods of Preparing Metal-Tagged Probes

In some aspects, the methods provided comprise providing a partially reduced antibody and reacting a sulfhydryl of the antibody with a maleimido attachment group of the polymer. The method can further comprise attaching a linker to the functional group of the polymer, wherein the linker is capable of binding a biomolecule. The method can further comprise covalently binding a biomolecule to the linker.

In some aspects, the polymer can be attached to a linker or it can be attached to a linker and a spacer.

In some aspects, the polymer comprises between about 1 to about 1000 (or more) atoms of an isotope as part of its structure.

In some aspects, a flexible linker/spacer at one end of the polymer may contain a thiol-reactive functional group such as a maleimide, and through this group can be linked to a binding reagent (for example an antibody). In some aspects, variations like attachment to primary amines of biomolecules or other methods of attachment known to persons skilled in the art can be used.

In some aspects, a binding reagent is coupled to a metal-tagged polymer using, e.g., partial or full reduction of disulfide bonds in an antibody or binding reagent. In some aspects, an antibody is partially reduced to not interfere with the antibody's overall structure. In some aspects, reduction can be performed using immobilized trialkylphosphine TCEP (Tris[2-carboxyethyl] phosphine hydrochloride) covalently linked to a beaded agarose support (Pierce). TCEP is an efficient reductant of alkyl disulfides over a wide range of pH and does not interfere with commonly used sulfhydryl-reactive reagents. In some aspects, the use of beads permits recovery of the reduced antibody or other affinity reagent by simple centrifugation from the reducing agent with subsequent separation from the beads.

In some aspects, polymer conjugated antibodies are purified to separate the antibody based on its large size of about 150 KDa (for an IgG antibody) from excess metallated labeling polymer (20-40 KDa) using gel filtration chromatography.

In some aspects, Protein A and Protein G are used to purify antibodies.

In some aspects, the metal atoms are added to the polymer tag at different steps during the production of the tagged biomolecule. For example, the metal of the tag can be added after conjugation of the antibody or other affinity reagent to the polymer. This strategy has several advantages. Conversion of an antibody-polymer conjugate into an antibody-metal-polymer conjugate can be done directly before an assay. A multitude of metal-tagged probes can be tagged with the same polymer conjugate under the same conditions. Further, the choice of metal or isotope to be used can be determined directly before the multiplexed experiment significantly increasing experimental flexibility. Also, the decoupling of both tagging stages allows a series of important independent control experiments in which the same antibody can be tagged with different metals. Finally, the selection of the internal standards is unhindered, and the relative sensitivity of the elemental analyzer can be effectively controlled.

The order of steps for the synthesis of the tagged biomolecule can be varied. For example, in some aspects, a metal is bound to the polymer prior to binding the polymer to an antibody.

In some aspects, a linker is bound to a polymer, a metal is bound to the polymer and subsequently an antibody is bound to the linker.

In some aspects, a linker is bound to the polymer, the linker is bound to an antibody and a metal is subsequently bound to the polymer.

In some aspects, the polymer metal tag may be attached to a biomolecule which is other than a binding reagent. For example, the polymer metal tag may be attached directly to an analyte, for example but not limited to a growth factor, cytokine or chemokine for studying kinetics of ligand-receptor interactions. In some aspects, a growth factor with a metal tag may be used as a probe to investigate the growth factor receptor abundance on a cell surface, analyze receptor dimerization and receptor internalization. In some aspects, two or more analytes may be analyzed in a multiplex reaction.

### VI. Methods of Using the Metal-Tagged Probes for Barcoding

In some aspects, provided is a method for the use of a metal-tagged probe described herein for barcoding a sample. In some aspects, the method comprises (i) incubating the metal-tagged probe with a sample comprising a cell, wherein the metal-tagged probe binds the cell; (ii) separating unbound metal-tagged probe from the bound metal-tagged probe; and (iii) analyzing the isotope bound to the metal-tagged probe attached to the cell by elemental analysis.

In some aspects, a n-choose-2 barcoding scheme is used to barcode multiple samples, e.g., resulting from multiple experimental conditions. In some aspects, multiple metal tags are used to barcode a single sample.

In some aspects, “n” denotes the number of different metal tags used in barcoding scheme and determines the barcoding depth. By using a n-choose-2 barcoding scheme (n*(n−1))/2 experimental conditions can be barcoded where two different metal tags are used per sample as a barcode. For example, using of 6-choose-2 scheme 15 different experimental conditions can be barcoded, pooled and processed simultaneously to overcome technical variations.

In some aspects, metal isotopes situated at the lower end of sensitivity spectrum (atom mass 75-152) are used for barcoding using antibodies that bind abundantly expressed antigens such as, e.g., CD45, B2M, CD298 and/or HLA-ABC expressed on hematopoietic cells.

In some aspects, metal isotopes with an atom mass between 153-208 are used for barcoding using antibodies that bind antigens with low abundance because such metals have higher detection sensitivities.

In some aspects, the barcoded samples are pooled, processed, and acquired on a CyTOF mass cytometer simultaneously to avoid variability due to sample processing.

In some aspects, different combinations of palladium, rhodium, yttrium, ruthenium, cadmium and tin isotopes are used to generate barcoding tags using antibodies against CD45, B2M, CD298 and HLA-ABC.

In some aspects, different combinations of rhenium, lead and mercury isotopes are used to generate barcoding tags using antibodies against antigens with low abundance on the cells of the sample to be interrogated.

In some aspects, antibodies against several different antigens can be tagged with the same metal to improve signal resolution (i.e., 106Cd CD45/B2M/CD298).

In some aspects, barcoding schemes are tailored to the use of a low number of barcodes, where the number of experimental conditions is low. Advantageously, the channels of a mass cytometer unused during analysis of such a low number of barcodes can be used for analyte assessment. For example, if only palladium, rhodium, yttrium and ruthenium isotopes are used for barcoding, cadmium and tin, lead, lanthanum, mercury and rhenium can be used to assess a higher number of analytes per single cell.

In some aspects, any of the metal tags disclosed herein can be used for generating barcoding reagents or for assessing analytes on cells.

In some aspects, the pooled samples are deconvoluted after sample acquisition. In some aspects, deconvolution is performed using Premessa R-package. (https://github.com/ParkerICI/premessa). In some aspects, de-barcoding parameters are adjusted to allow optimal barcode separation. The de-barcoding parameters may vary between different experimental conditions. In some aspects, after sample cleanup, the deconvoluted samples are subjected to downstream high-dimensional analysis.

In some aspects, the metal-tagged probes described herein are used for barcoding a sample and further metal-tagged probes described herein are used for analyte assessment of the barcoded sample.

In some aspects, the method comprises (i) incubating a sample comprising cells with at least one barcoding reagent comprising a plurality of barcodes of metal-tagged probes that bind the cells of the sample; (ii) separating unbound metal-tagged probes from the bound metal-tagged probes; (iii) incubating the sample comprising the barcoded cells with at least one further reagent comprising a plurality of metal-tagged probes that bind molecules of interest or analytes on the cells of the sample, wherein the metal tags of the further reagent comprise isotopes that are of different atomic mass than the metal tags of the barcoding reagent; (vi) separating unbound metal-tagged probes from the bound metal-tagged probes; and (iii) analyzing the isotopes bound to the cells through the metal-tagged probes using a mass cytometer.

In some aspects, the method comprises a multiplex analysis of two or more analytes. In some aspects, the method comprises (i) incubating a sample comprising cells with at least one barcoding reagent comprising a plurality of barcodes of metal-tagged probes that bind the cells of the sample; (ii) separating unbound metal-tagged probes from the bound metal-tagged probes; (iii) incubating the sample comprising the barcoded cells with multiple further reagents each comprising a plurality of metal-tagged probes that bind multiple molecules of interest or analytes on the cells of the sample, wherein the metal tags of the further reagents comprise isotopes that are of different atomic mass than the metal tags of the barcoding reagent; (vi) separating unbound metal-tagged probes from the bound metal-tagged probes; and (iii) analyzing the isotopes bound to the cells through the metal-tagged probes using a mass cytometer.

In some aspects, a multiplex analysis can, for example, use seven barcodes for barcoding 21 different samples. In some aspects, spill over of isotopes into neighboring mass cytometer channels is determined and combinations of isotopes less spillover in their detection channels from neighboring channels are used for analysis. In some aspects, a multiplex analysis uses metal-tag probes with low sensitivity isotopes with antibodies that bind abundant antigens. In some aspects, a multiplex analysis uses metal-tag probes with high sensitivity isotopes with antibodies that bind antigens with low abundance on the cells of the sample interrogated.

In some aspects, for example, 11-choose-2 or 12-choose-2 barcoding schemes are used to pool and analyze up to 55 or 66 samples simultaneously.

In some aspects, the barcoding schemes described herein are used on live cells for the assessment of cell surface analytes.

In some aspect, the barcoding schemes described herein are used on fixed and permeabilized cells for assessment of intracellular analytes.

In some aspects, the methods and barcoding reagents described herein enable detection of an analyte on a cell at a level of increased sensitivity compared to a metal-tagged probe that does not comprise a metal tag as described herein.

In some aspects, the methods described herein are used to stain cells in a tissue section sample.

### VII. Kits

In some aspects, provided are kits for the preparation of metal tags described herein, comprising at least one of the following: a polymer comprising at least one metal-binding group which comprises at least one metal atom or is capable of binding at least one metal atom and a metal atom. In some aspects, the polymer comprising a functional group that allows the polymer to be attached to a biomolecule, a linker, or a spacer. In some aspects, the kit further comprises a biomolecule such as an antibody and a reducing agent to partially reduce the sulfhydryl groups of the antibody.

In some aspects, the kit further comprises a metal solution, reagents for the attachment of a linker, spacer or biomolecule to the polymer; reagents for attachment of a functional group to the linker or the spacer; reagents for attachment of a metal to the polymer; buffers; and instructions for preparing the metal tag, conjugating the metal tag to a biomolecule including an antibody, and instructions for conducting a multiplex barcoding scheme analysis.

In some aspects, the polymer for any of the above kits can be a homopolymer of acrylamide, a copolymer of acrylamides, a methacrylamide, an acrylate ester, a methacrylate ester, an acrylic acid or a methacrylic acid.

In some aspects, the reagents can include at least one of the following: TCEP (tri(2-carboxyethyl)phosphine), Ligand-Polymer-Linker-Spacer Conjugate, phosphate buffer, TBS (tris-buffered saline), EDTA (Diaminoethanetetraacetic acid), ammonium acetate buffer, antibodies, metal salt solutions, blocker buffers, washing buffers, FBS (fetal bovine serum), DMEM (Dulbecco's Modified Eagle's Medium), SA (bovine serum albumin), dithiothreitol, bismaleimide, and DMF (dimethylformamide).

### Examples

### Materials and Methods

### Sample Preparation.

Peripheral blood samples were obtained from healthy donors. PBMCs were isolated by density-gradient centrifugation using lymphocyte separation medium (Lymphoprep; Axis Shield, Oslo, Norway). Isolated PBMCs were cryopreserved in freezing media containing 10% dimethyl sulfoxide and 90% fetal bovine serum. Frozen PBMCs were thawed rapidly in 37° C. water bath and half-thawed samples were transferred to pre-warmed culture media (90% RPMI 1640) containing 2 mM GlutaMAX TM-I, 1% Penicillin-Streptomycin and 10% fetal bovine serum) and supplemented with 50 IU-ml benzonase (Sigma-Aldrich, St. Louis, MO). Thawed PBMCs were washed once and incubated overnight at 37° C. with air containing 5% CO2 before staining.

### Antibody Conjugation.

Antibodies, corresponding metal tags, and related information are shown in Table 1. Unlabeled antibodies were purchased in carrier-free form and conjugated to lanthanides and indium isotopes using Maxpar® X8 polymer per manufacturer's instructions (Fluidigm®, San Francisco, CA). Lanthanides and Indium isotopes were obtained from Fluidigm® and Trace Sciences, respectively. Monoisotopic Indium salts were dissolved in distilled H₂O to 1 M stock solution, then further diluted to 50 mM in L buffer (Fluidigm®, San Francisco, CA) and loaded onto Maxpar® X8 polymer (Fluidigm®, San Francisco, CA). As for conjugation of unlabeled antibodies to monoisotopic cisplatin containing 194Pt or 198Pt isotopes, 100 μg of unlabeled carrier-free antibody was washed with R buffer in a 50 kilodalton (kDa) spin filter column (EMD Millipore), reduced in R buffer using 4 mM final concentration of TCEP for 30 min at 37° C. and then washed twice with C buffer. The reduced antibody was resuspended in C buffer, monoisotopic cisplatin compounds containing either 194Pt or 198Pt (Fluidigm®, San Francisco, CA) was added to a final concentration of 100 μM and total volume was adjusted to 400 μl with C buffer. The antibody-cisplatin mixture was incubated for two hours at 37° C. and washed four times with W buffer (Fluidigm®, San Francisco, CA). Antibody concentration was determined by absorbance reading 280 nm using NanoDrop™ 2000 (Thermo Fisher Scientific™, Waltham, MA). All conjugated antibodies were then diluted to 0.5 mg/ml final concentration either in PBS-based antibody stabilization solution or Low Cross-Buffer® (Candor Bioscience® GmbH, Wangen, Germany) supplemented with 0.05% sodium azide (Sigma-Aldrich®, St. Louis, MO). Serial titration experiments were performed to determine the concentration giving the optimal signal-to-noise ratio for each antibody.

TABLE 1
Source information for antibodies and metal tags described in the Examples.

| Isotope | Metal | Antigen | Clone | Polymer | Vendor | Category |
|---|---|---|---|---|---|---|
| 104 | Pd | CD45 | HI30 | MCP9/mDOTA | BioLegend | Barcoding |
| 105 | Pd | CD45 | HI30 | MCP9/mDOTA | BioLegend | Barcoding |
| 106 | Cd/Pd | CD45 | HI30 | MCP9/mDOTA | BioLegend | Barcoding |
| 108 | Pd | CD45 | HI30 | MCP9/mDOTA | BioLegend | Barcoding |
| 110 | Cd/Pd | CD45 | HI30 | MCP9/mDOTA | BioLegend | Barcoding |
| 111 | Cd | CD45 | HI30 | MCP9 | BioLegend | Barcoding |
| 112 | Cd | CD45 | HI30 | MCP9 | BioLegend | Barcoding |
| 113 | In/Cd | CD8/CD45 | RPA-T8/HI30 | mDTPA | BioLegend | Surface |
| 114 | Cd | CD45 | HI30 | MCP9 | BioLegend | Barcoding |
| 115 | In | CD3 | HI30 | mDTPA | BioLegend | Surface |
| 116 | Cd | CD45 | HI30 | MCP9 | BioLegend | Barcoding |
| 141 | Pr | CCR6 | G034E3 | mDTPA | Fluidigm | Surface |
| 142 | Nd | CD27 | M-T271 | mDTPA | BD | Surface |
| 143 | Nd | CD56 | NCAM16.2 | mDTPA | BD | Surface |
| 144 | Nd | CD26 | BA5b | mDTPA | BioLegend | Surface |
| 145 | Nd | NKG2A | REA110 | mDTPA | Miltenyi | Surface |
| 146 | Nd | CD49F | GoH3 | mDTPA | BD | Surface |
| 147 | Sm | CD127 | REA614 | mDTPA | Miltenyi | Surface |
| 148 | Nd | CCR2 | REA264 | mDTPA | Miltenyi | Surface |
| 149 | Sm | CCR4 | 205410 | mDTPA | R&D | Surface |
| 150 | Nd | CD28 | CD28.2 | mDTPA | BioLegend | Surface |
| 151 | Eu | Tbet | O4-46 | mDTPA | BD | Intracellular |
| 152 | Sm | TIGIT | MBSA43 | mDTPA | Thermofisher | Surface |
| 153 | Eu | CD45RA | HI100 | mDTPA | BioLegend | Surface |
| 154 | Sm | Ki-67 | 20Raj1 | mDTPA | Thermofisher | Intracellular |
| 155 | Gd | CCR7 | G043H7 | mDTPA | BioLegend | Surface |
| 156 | Gd | TIM3 | F38-2E2 | mDTPA | BioLegend | Surface |
| 157 | Gd | CD19 | HIB19 | mDTPA | BioLegend | Surface |
| 158 | Gd | 2B4 | C1.7 | mDTPA | BioLegend | Surface |
| 159 | Tb | ICOS | C398.4A | mDTPA | BioLegend | Surface |
| 160 | Gd | PD-1 | EH12.2H7 | mDTPA | BD | Surface |
| 161 | Dy | CXCR3 | G025H7 | mDTPA | BioLegend | Surface |
| 162 | Dy | Eomes | WD1928 | mDTPA | Thermofisher | Intracellular |
| 163 | Dy | CD137 | 4B4-1 | mDTPA | BD | Surface |
| 164 | Dy | CTLA-4 | BN13 | mDTPA | Bioxcell | Surface |
| 165 | Ho | CD25 | 2A3 | mDTPA | BD | Surface |
| 166 | Er | LAG3 | REA351 | mDTPA | Miltenyi | Surface |
| 167 | Er | Perforin | B-D48 | mDTPA | Diaclone | Intracellular |
| 168 | Er | CD160 | 688327 | mDTPA | R&D | Surface |
| 169 | Tm | CD69 | FN50 | mDTPA | BD | Surface |
| 170 | Er | CD39 | A1 | mDTPA | Bio-Rad | Surface |
| 171 | Yb | CD161 | 191B8 | mDTPA | Miltenyi | Surface |
| 172 | Yb | KLRG1 | 13F12F2 | mDTPA | Thermofisher | Surface |
| 173 | Yb | CD95 | DX2 | mDTPA | BD | Surface |
| 174 | Yb | GITR | DT5D3 | mDTPA | Miltenyi | Surface |
| 175 | Lu | CD4 | RPA-T4 | mDTPA | BioLegend | Surface |
| 176 | Yb | CD38 | REA572 | mDTPA | Miltenyi | Surface |
| 191 | Ir | DNA | N/A | N/A | Fluidigm | Other |
| 193 | Ir | DNA | N/A | N/A | Fluidigm | Other |
| 194 | Pt | CD57 | HCD57 | N/A | BioLegend | Surface |
| 195 | Pt | live-dead | N/A | N/A | Enzo | Other |
| 196 | Pt | live-dead | N/A | N/A | Enzo | Other |
| 198 | Pt | HLA-DR | L243 | N/A | BioLegend | Surface |
| 209 | Bi | CLA | HECA-452 | mDTPA | BD | Surface |

### Generation of Cd and Pd Barcoding Reagents.

Seven Cd isotopes were acquired from Fluidigm® and were conjugated to CD45 antibody (Clone HI30; Biolegend®, San Diego, CA) using Maxpar® MCP9 antibody labeling kit (Fluidigm®, San Diego, CA) per manufacturer's instructions. Briefly, 13 μl of Cd isotopes were loaded onto 200 μg of MCP polymer suspended in 87 μl of L buffer and incubated for one hour at 37° C. in water bath. Following incubation, Cd loaded MCP9 polymer was washed twice in L and once in C buffer on 3 kDa spin filter and suspended in 60 μl C-buffer.

One hundred μg of CD45 or B2M antibodies was washed twice in R buffer and then reduced in 4 mM TCEP in 100 μl R buffer for 30 min at 37° C. The reduced antibody was washed twice in C-buffer. Cd-loaded MCP polymer was transferred to the 50 kDa filter containing the reduced antibody.

Reduced antibody/Cd-loaded polymer mixture was incubated for 90 min at 37° C. After incubation, antibodies were transferred to 100 kDa filter and washed four times for five min at 5000×g. Antibody concentration was determined based on absorption at 280 nm using NanoDrop® 2000 (Thermo Fisher Scientific®, Waltham, MA). All conjugated antibodies were diluted to 0.5 mg/ml final concentration in HRP-Protector™ (Candor Bioscience® GmbH, Wangen, Germany).

Five monoisotopic palladium nitrate compounds, 104Pd, 105Pd, 106Pd, 108Pd, and 110Pd, were sourced from Trace Sciences® and dissolved in hydrochloric acid (HCl) to 50 mM concentration. A protocol previously published by Han, G., Spitzer, M. H., Bendall, S. C., Fantl, W. J. & Nolan, G. P. Metal-isotope-tagged monoclonal antibodies for high-dimensional mass cytometry. Nat. Protoc. 13, 2121-2148, 2018, was adapted. Maleimido-mono-amide-DOTA (mDOTA, Macrocyclics™, Dallas, TX) was used to chelate Pd isotopes. Briefly, mDOTA was dissolved in double-distilled water (ddH2O) at 25 mM concentration. Palladium isotopes were mixed with mDOTA at 1:1.2 molar ratio. The mixture was immediately snap-frozen and lyophilized overnight. Lyophilized Pd loaded mDOTA complex was dissolved in dimethyl sulfoxide to 10 mM concentration. One microliter of Pd-loaded DOTA was added to partially reduced antibody suspended in 100 μl C-buffer. The mixture was then incubated for at least one h at 37° C. Zeba 7 kDA spin columns (Thermofisher Scientific®, USA) were used for antibody purification. Briefly, Zeba 7 kDA spin columns were spun to remove storage solution and then washed twice with 300 μl W buffer (Fluidigm®). Pd-loaded mDOTA and antibody mixture was transferred to a Zeba 7 kDA spin column and spun at 1,500×g for two min. The antibody concentration was measured based on absorption readout at 280 nm. For solvent removal before suspending in antibody stabilizing solution the flow-through was then transferred to a new 50 kDa spin filter and spun at 12,000×g for five min.

Antibodies tagged with Pd-loaded mDOTA were diluted to 0.5 mg/ml in PBS-based antibody stabilization solution or LowCross-Buffer® (Candor Bioscience® GmbH, Wangen, Germany) supplemented with 0.05% sodium azide (Sigma-Aldrich®, St. Louis, MO).

### Antibody Conjugation Using Pd-Loaded MCP9 Polymers.

Two Pd isotopes, 106Pd and 110Pd, overlap with 2 Cd isotopes, 106Cd and 110Cd, having similar mass weights. Hence, three monoisotopic palladium nitrate compounds, 104Pd, 105Pd and 108Pd, were previously dissolved in HCl to 50 mM concentration to load onto DOTA and ITCBE chelators. Pd isotopes suspended in 5 N HCl were lyophilized overnight and suspended in nitric acid to generate Pd(NO3)2 salts. Isotopically enriched Pd nitrate solution was lyophilized overnight and suspended in water to 50 mM concentration.

A similar approach was used for a Cd-MCP9 antibody conjugation protocol to load Pd metals onto MCP9 polymers. Briefly, 13 μl of monoisotopic Pd isotope was loaded onto 200 μg of MCP polymer and washed twice with L and once with C buffer after incubation at 37° C. for one hour. Pd-loaded MCP9 polymer was then mixed with reduced CD45 antibody and incubated for 90 min at 37° C. After incubation, antibodies were transferred to 100 kDa filter and washed using W buffer (Fluidigm®, San Diego, CA). Antibody concentration was determined based on absorption at 280 nm using NanoDrop® 2000 (Thermo Fisher Scientific®, Waltham, MA) All conjugated antibodies were then diluted to 0.5 mg/ml final concentration in HRP-protector (Candor Bioscience® GmbH, Wangen, Germany).

Similar approaches can be used to conjugate the following antibodies to metal tags described herein: anti-CD19, anti-CD3, anti-CD4, anti-CD8, anti-TNFα, anti-IFN-γ, anti-CD16, anti-CD33, anti-CD36, anti-phosphoSTAT5, anti-IL2, anti-CD28, anti-CD14, anti-T-bet, anti-CD66b, anti-B2M, anti-CD20, anti-HLA-DR, anti-CD14, anti-CD56, anti-CD294, anti-CD38, anti-CD11c, anti-CD57, anti-CD123, anti-TCRγδ, anti-CD161, anti-CCR7, anti-CD45RA, anti-CD27, anti-CD45RO, anti-CD25, anti-CCR5, anti-CCR4, anti-CXCR3, and anti-CCR6, anti-CD26, anti-KG2A, anti-CD49F, anti-CD127, anti-CCR2, anti-TIGIT, anti-Ki-67, anti-TIM3, anti-2B4, anti-ICOS, anti-PD-1, anti-Eomes, anti-CD137, anti-CTLA-4, anti-LAG3, anti-Perforin, anti-CD160, anti-CD69, anti-CD39, anti-KLRG1, anti-CD95, anti-GITR, and anti-CLA, anti-CD73, Anti-CD298, anti-CD98, anti-G6PD, anti-GAPDH, anti-CD15S, anti-pS6, anti-pAKT, anti-MEK1/2, anti-pERK1/2, anti-phosphoSTAT3, anti-phosphoSTAT1, anti-phosphoSTAT4, anti-phosphoSTAT6, anti-p4EBP1, anti-GLUT1, anti-H3K27, anti-c-myc, anti-granzyme A, anti-granzyme B, anti-granzyme K, anti-granzyme M, anti-Helios, anti-RUNX3, anti-p53, anti-HIF1a, anti-p-NRF2, anti-Bcl-2, anti-Mcl-1, anti-Bcl-xL, anti-BIM, anti-Bax, anti-CD90, anti-CD96, anti-OX40, anti-MIP1a, anti-MIP1b, anti-IL4, anti-IL5, anti-IL17A, anti-IL6, anti-MCP1, anti-CD112, anti-CDD115, anti-CD34, anti-CD11b, anti-CD11a, anti-CD141, anti-CD105, anti-CD1a, anti-CD1c, anti-CD235a, anti-YTHDF2, anti-CD41a, anti-CD71, anti-Siglec7, anti-CD30, anti-CD61, anti-RANTES, anti-MCT-1, anti-MCT4, anti-CD80, anti-CD83, anti-CD86, anti-KIR3DL1, anti-KIR3DL2, anti-KIR2DL1, anti-NKG2C, anti-CD94, anti-NRF2, anti-CDT1, anti-pRB, anti-CyclinB1, or anti-BrU.

### Capture Bead Labeling.

Anti-mouse Ig kappa antibody capture beads (BD Biosciences®, San Jose, CA) were used to assess and compare signal intensity for Cd and Pd labeled antibodies. Briefly, equal amounts of antibody capture beads were stained with Cd and Pd-labeled antibodies and incubated at room temperature for 20 min. Following incubation, capture beads were washed twice with 0.5% bovine serum albumin (BSA)/PBS solution (staining buffer) and then fixed using 1.6% paraformaldehyde (PFA) (Electron Microscopy Sciences, Hatfield, PA) for one hour at room temperature (RT). Fixed beads were washed twice with 0.5% BSA/PBS and twice with ddH2O.

The beads labeled with single Cd or Pd-tagged CD45 antibody were suspended in 500 μl of ddH2O and acquired individually on a Helios™ mass cytometer (Fluidigm®, San Diego, CA). Single events were chosen following gating on event length and Cd or Pd channels of interest using FlowJo version 10.6 (TreeStar; Ashland, OR)

### Live-Cell Barcoding.

PBMCs including up to 2×10⁶ cells were stained with different combinations of Cd i.e., 106Cd, 110Cd, 111Cd, 112Cd, 113Cd, 114Cd, and 116Cd) and Pd (i.e., 104Pd, 105Pd, and 108Pd) tagged CD45 antibodies at a final concentration of 2.5 μg/ml per antibody and incubated at RT for 30 min. A 10-choose-2 barcoding scheme was utilized allowing to barcode up to 45 different experimental conditions with doublet filtering scheme. Samples, each tagged with a unique barcode, were washed three times with staining buffer. Washed samples were then pooled and incubated with 5 μM of natural abundance cisplatin (Enzo, Farmingdale, NY) or 1 μM of monoisotopic cisplatin-196 Pt (Neonest AB, Stockholm, Sweden) for one min at RT.

### Sample Staining.

Pooled samples were blocked with 5 μl of human Trustain FcX™ (Biolegend®, San Diego, CA), Fc receptor blocking solution, for 10 min at RT. Cells were then stained with a collection of T-cell focused antibodies and incubated 30 min at RT. The antibody panel is presented in Table 1. Cells were washed twice with staining buffer after incubation, fixed in 1.6% PFA for 10 min at RT, permeabilized in 90% methanol at −20° C. for 60 min. Permeabilized cells were washed twice with staining buffer and stained with intracellular antibodies. Cells were washed twice with staining buffer following incubation for 30 min at 4° C., resuspended in intercalator solution (1.6% PFA in PBS with 125 nM iridium nucleic acid intercalator) and incubated at 4° C. overnight.

### Sample Acquisition and Data Processing.

Samples were washed twice with cell staining buffer, resuspended in 1 ml of Milli-Q® dH2O, filtered through 35 μm nylon mesh (cell strainer cap tubes, BD, San Jose, CA), counted and washed before sample introduction. Samples were suspended at a concentration of 0.6×106/ml in Milli-Q® ddH2O supplemented with 10% EQ Four Element Calibration Beads (Fluidigm®, San Francisco, CA) and acquired at 300 events/second on Helios™ instrument using CyTOF Software version 6.7.1016 (Fluidigm®, San Francisco, CA). CyTOF® data were normalized based on signal drift over time using CyTOF® Software.

### De-Barcoding and Data Analysis.

Deconvolution of pooled sample sets was performed using Premessa R-package. (https://github.com/ParkerICI/premessa). De-barcoding parameters were adjusted to allow optimal barcode separation. Initial data processing of deconvoluted samples was performed by using Flowjo version 10.6. Calibration beads were gated out and singlets were chosen based upon DNA content and event length. Dead cells were excluded by selecting cells with low cisplatin uptake. Desired populations of interest are gated on and then exported for downstream analyses. T-Distributed Stochastic Neighbor Embedding (t-SNE)36 and uniform manifold approximation and projection (UMAP)37 for dimension reduction was used, and FlowSOM38 and PhenoGraph clustering algorithms in Cytofkit39 using R software version 3.6.3 (R Core Team, 2020) was used. R: a language environment for statistical computing. R Foundation for Statistical Computing, Vienna, Austria (URL https://www.R-project.org). The analyses were performed by using all the markers except for the parameters used for manual gating to identify and export downstream subpopulations.

### Example 1—Generation of Metal Tags

MCP9 polymers (Fluidigm®, San Francisco, CA), were used to chelate seven bivalent Cd isotopes to generate low-sensitivity metal-tagged antibodies with improved signal-to-noise ratio. CD45, which is expressed at varying abundance across different subsets of hematopoietic cells was used as antigen to generate barcoding reagents.

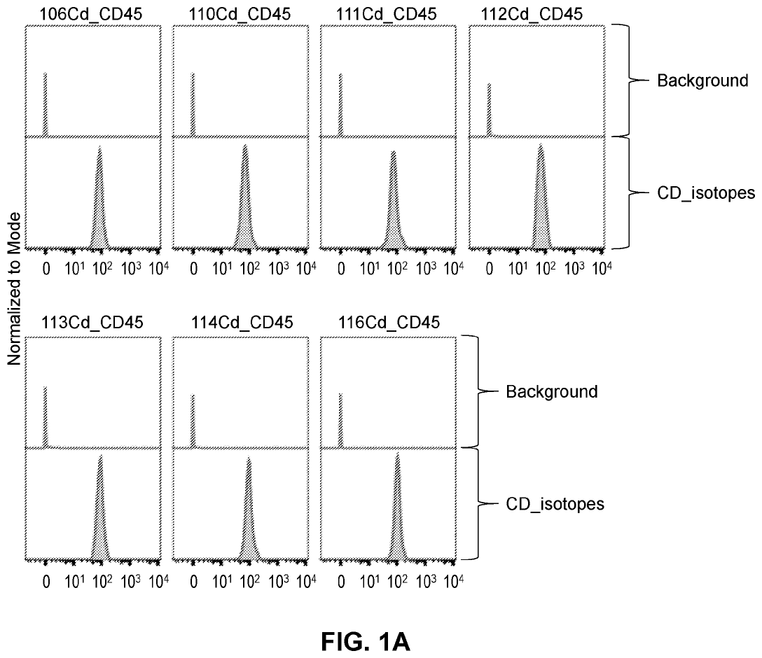

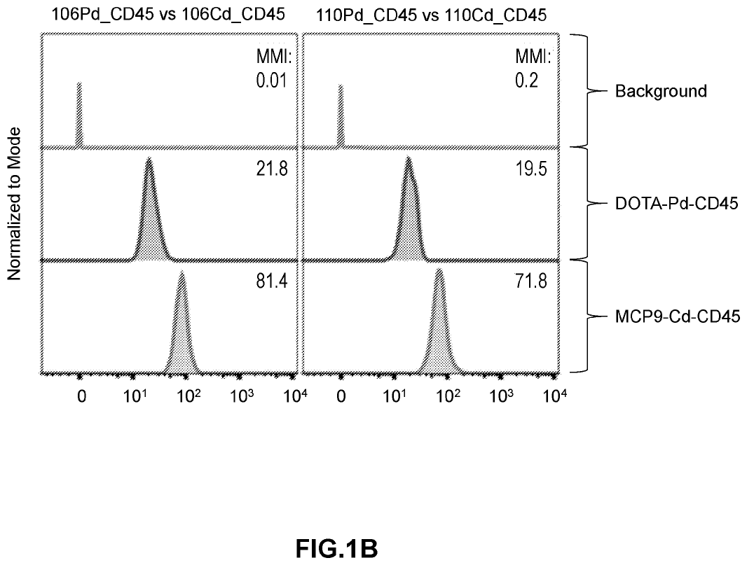

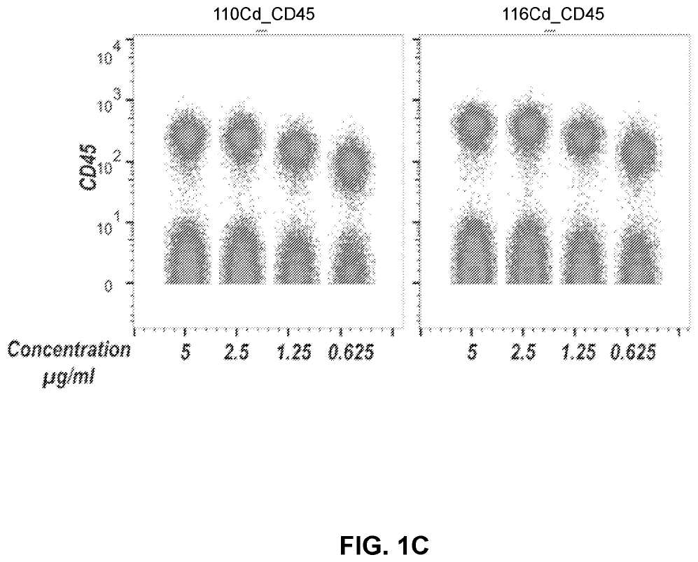

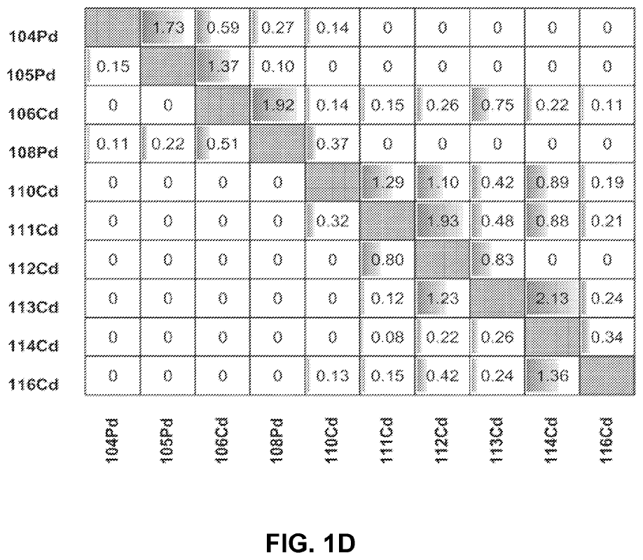

Seven CD45-based barcoding reagents were generated by pairing CD45 antibody (HI30) with seven MCP9-loaded Cd isotopes. The antibody yield after conjugation ranged from 60 to 70%. Five CD45-mDOTA-Pd conjugates (i.e., 104Pd, 105Pd, 106Pd, 108Pd, and 110Pd) were generated for comparison and antibody recovery after tagging with Pd-loaded mDOTA monomers was approximately 80 to 90%. The difference in recovery was due to the necessity to utilize different antibody purification methods. The polymeric bifunctional chelator MCP9 could chelate and capture more bivalent ions compared to monomeric bifunctional chelator mDOTA, and thus generated higher signal intensities. Initially, antibody-labeled capture beads were used to estimate the performance of the Cd-tagged CD45 antibodies in comparison to CD45 antibodies labeled with Pd isotopes loaded onto mDOTA. Capture beads were labeled with equal amounts of CD45 antibodies tagged with MCP9-loaded Cd and mDOTA-loaded Pd isotopes. The capture beads labeled with CD45-tagged with 7 Cd isotopes generated appreciable signal intensities (FIG. 1A). Having two cadmium isotopes, 106Cd and 110Cd, overlapping with two Pd isotopes, 106Pd and 110Pd, enabled the direct comparison of Cd isotopes loaded onto MCP9 polymer with corresponding Pd isotopes with similar mass loaded onto mDOTA monomer. Signal intensities associated with 106Cd and 110Cd-CD45 were significantly higher compared to 106Pd and 110Pd-CD45 antibodies, indicating that MCP9 polymers were capable of chelating a higher number of bivalent metals than the mDOTA monomers and provided superior signal resolution (FIG. 1B).

Further barcoding reagents were generated by loading different isotopes on either MCP9 polymer dissolved in L buffer from Fluidigm®, X8 polymer dissolved in water, or X8 polymer dissolved in 3% nitric acid.

For example, yttrium (III) chloride hydrate or yttrium (III) nitrate hydrate were dissolved in ddH₂O at pH 6 and loaded onto MCP9 polymer. The yttrium-MCP9 tags were spun in a 3K filter with polyethersulfone membrane to wash off excess metal.

Ruthenium (III) chloride hydrate was dissolved in 3% nitric acid and loaded onto X8 polymer in the presence of 3% nitric acid. The ruthenium-X8 tag was spun in a 3K filter with polyethersulfone membrane to wash off excess metal.

Rhodium (III) chloride hydrate was dissolved in ddH₂O at pH 6 and loaded onto MCP9 polymer. The rhodium-MCP9 tag was spun in a 3K filter with polyethersulfone membrane to wash off excess metal.

Palladium (II) nitrate hydrate was dissolved in ddH₂O at pH 6 and loaded onto MCP9 polymer. The palladium-MCP9 tag was spun in a 3K filter with polyethersulfone membrane to wash off excess metal.

Cadmium (II) nitrate was dissolved in Fuidigm®'s L buffer and loaded onto MCP9 polymer. The cadmium-MCP9 tag was spun in a 3K filter with polyethersulfone membrane to wash off excess metal.

Tin (II) chloride hydrate was dissolved in 5% nitric acid and loaded onto MCP9 polymer. The tin-MCP9 tag was spun in a 3K filter with polyethersulfone membrane to wash off excess metal.

Indium (III) chloride was dissolved in ddH₂O at pH 6 and loaded onto X8 polymer. The indium-X8 tag was spun in a 3K filter with polyethersulfone membrane to wash off excess metal.

Mercury (II) nitrate hydrate was dissolved in 5% nitric acid and loaded onto MCP9 polymer. The mercury-MCP9 tag was spun in a 3K filter with polyethersulfone membrane to wash off excess metal.

Lead (II) nitrate was dissolved in ddH₂O at pH 6 and loaded onto MCP9 polymer. The lead-MCP9 tag was spun in a 3K filter with polyethersulfone membrane to wash off excess metal.

Rhenium (III) chloride was dissolved in 3% nitric acid and loaded onto X8 polymer, which had been dissolved in ddH₂O. The rhenium-X8 tag was spun in a 3K filter with low-binding regenerated cellulose to wash off excess metal because the rhenium reacted with the polyethersulfone membrane.

Lanthanum (III) nitrate hydrate was dissolved in ddH₂O at pH 6 and loaded onto MCP9 polymer. The lanthanum-MCP9 tag was spun in a 3K filter with polyethersulfone membrane to wash off excess metal.

### Example 2—Cadmium Labeling of CD45 Antibodies Using the MCP9 Polymer and Use of Cd-MCP9-CD45 Probes

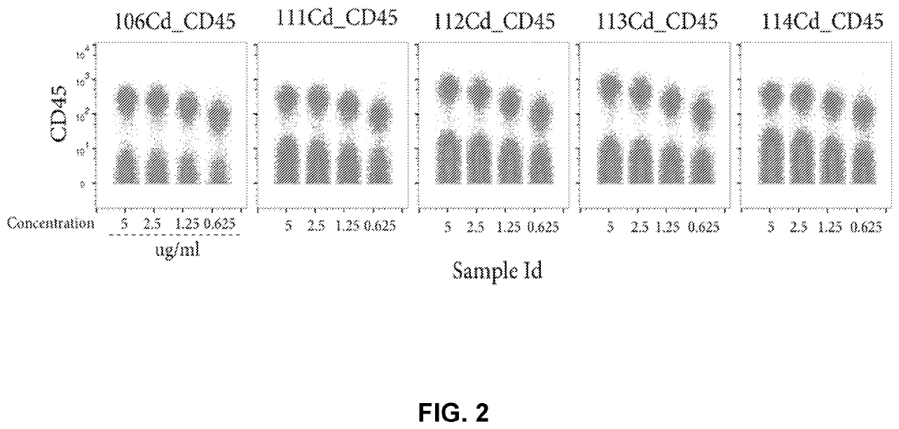

Peripheral blood mononuclear cells (PBMCs) from healthy donors were labeled with individual Cd-CD45 antibodies to assess if Cd signal intensities measured by capture beads would translate to superior single intensities and signal-to-noise ratios when PBMCs were used for labeling. Seven Cd-tagged CD45 antibodies were serially diluted separately and individual samples labeled with identical concentrations of different Cd-tagged CD45 antibodies were pooled before acquisition. Staining with a fixed 2.5 μg/ml concentration produced acceptable signal-to-noise ratios for the majority of antibodies (FIG. 1C and FIG. 2). FCS files were concatenated after separate acquisition of each file and concatenated FCS files displayed serial dilutions for 110Cd and 116Cd isotopes tagged with CD45 (FIG. 1C) and serial dilutions for 106Cd, 111Cd, 112Cd, 113Cd, and 114Cd tagged with CD45 (FIG. 2). Background noise due to feedback from neighboring channels was observed when samples were stained separately with individual barcoding tags and pooled before sample acquisition. This could potentially impinge on barcoding depth if a channel receives signals from other channels. For example, the 112Cd, 113Cd, 114Cd, and 116Cd channels received spillover from multiple channels (FIG. 1D), resulting in increased background noise (FIG. 1C and FIG. 2).

The results indicated that MCP9 polymers chelated higher numbers of metals relative to their monomeric counterparts, and provided markedly improved signal-to-noise ratios for metals situated at the lower end sensitivity spectrum.

### Example 3—Pd Labeling of CD45 Antibodies Using the MCP9 Polymer and Use of Pd-MCP9-CD45 Probes

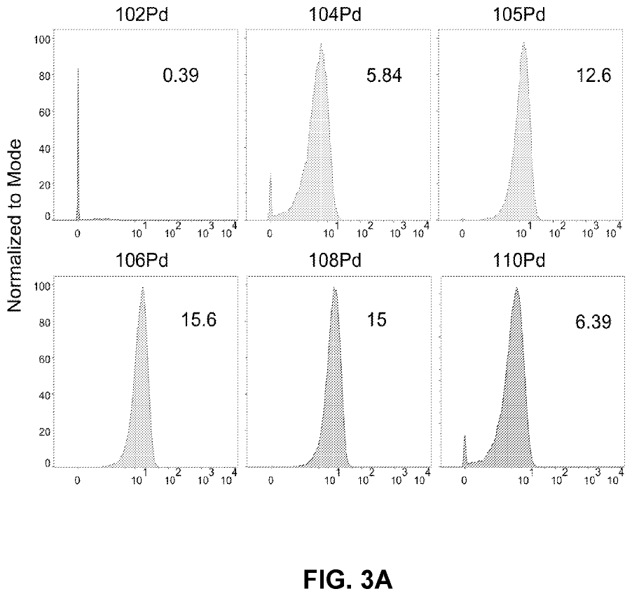

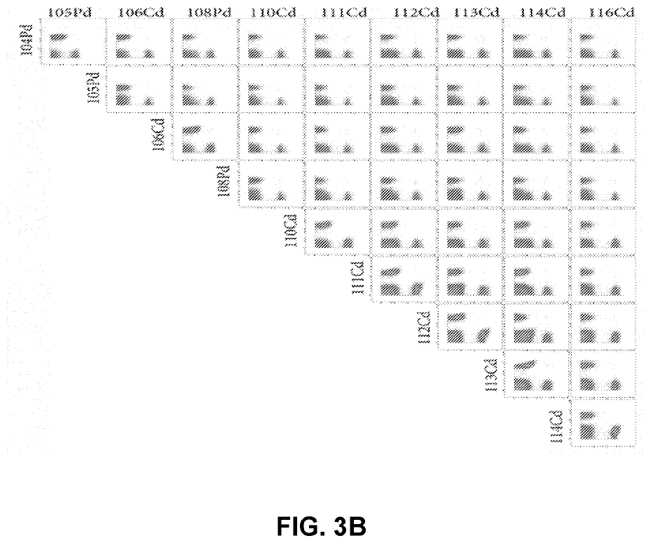

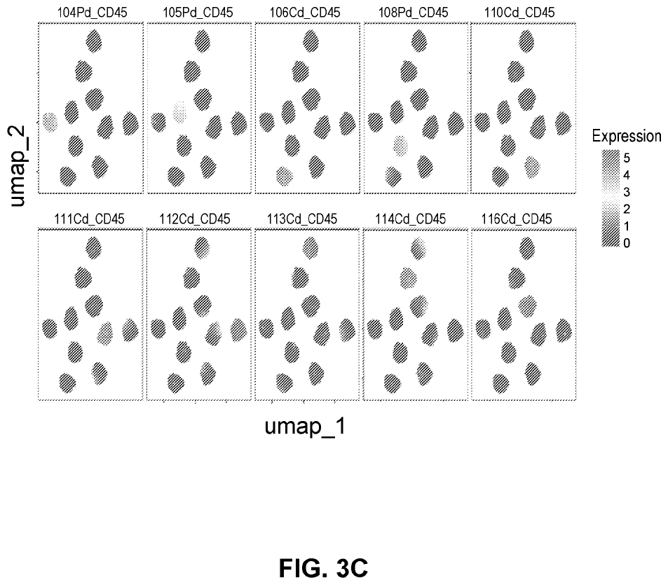

Initially natural abundance Pd(NO₃)₂ (palladium nitrate) was dissolved in double distilled water (ddH2O) to 50 mM and loaded onto MCP9 polymers. PBMCs were stained with CD45 tagged to MCP9-loaded natural abundance Pd and signal intensities observed demonstrated that MCP9 successfully captured Pd ions (FIG. 3A).

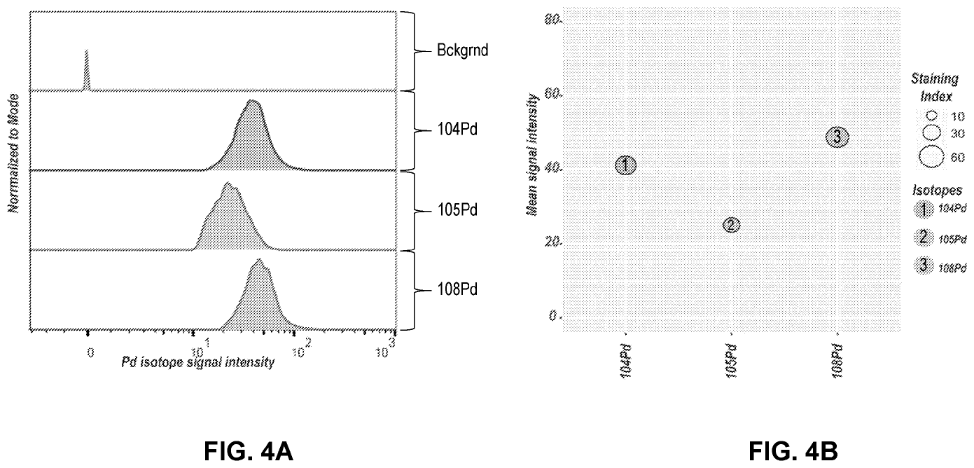

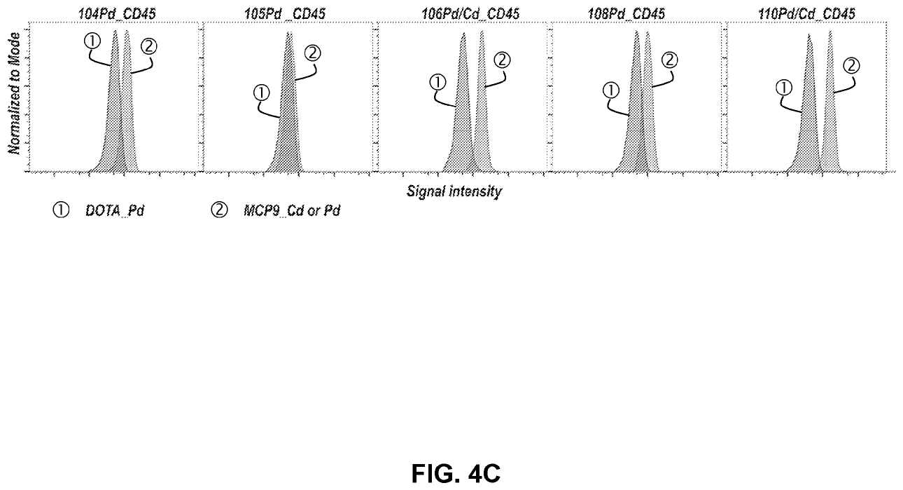

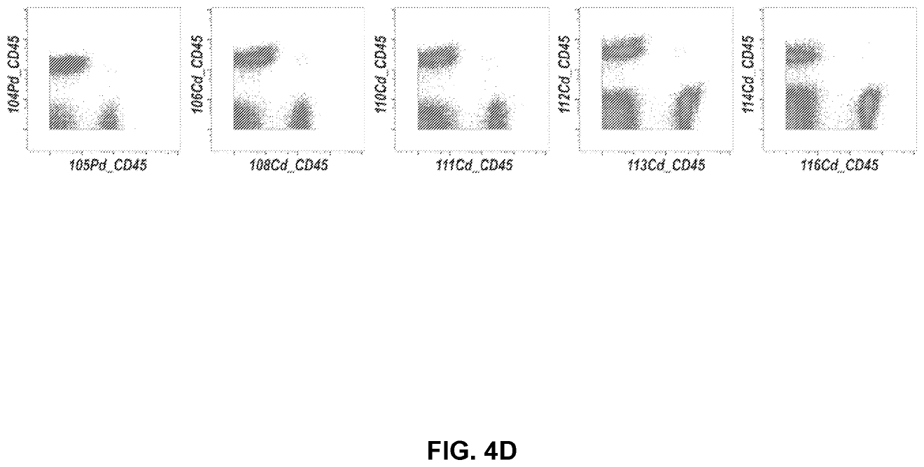

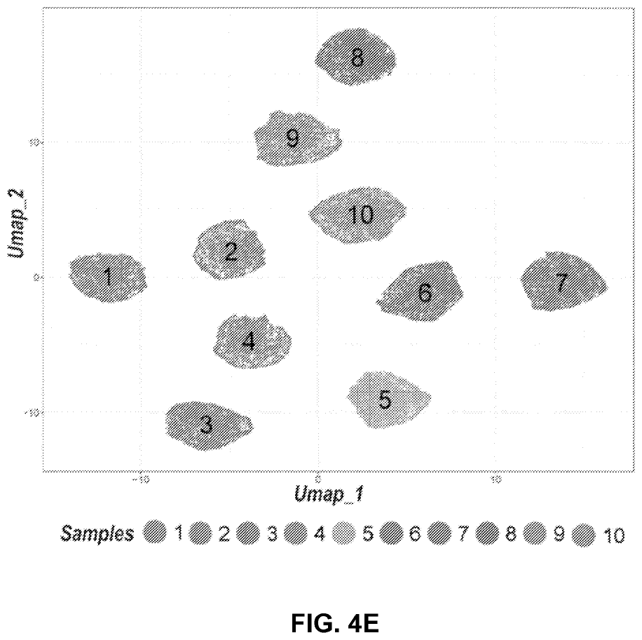

Next, three Pd isotopes, 104Pd, 105Pd, and 108Pd were loaded onto MCP9, Pd-MCP9 complexes were tagged to CD45 antibodies, and antibody capture beads were used to assess metal loading and antibody tagging efficiency. Capture beads were labeled with equal amounts of MCP9-loaded Pd isotopes tagged with CD45. MCP9 polymers were able to successfully capture Pd metals (FIG. 4A). A bubble plot showed mean signal intensities and staining indices for 104Pd, 105Pd, and 108Pd (FIG. 4B), Five Pd isotopes, 104Pd, 105Pd, 106Pd, 108Pd and 110Pd were loaded onto mDOTA. For comparison three Pd, 104Pd, 105Pd, and 108Pd, and two Cd isotopes, 106Cd and 110Cd, were loaded onto MCP9 polymer and conjugated to CD45 antibody. PBMCs from healthy donors were stained with 10 different MCBs, 5 mDOTA-based and 5 MCP9-based, at a concentration of 2.5 μg/ml for each antibody. Pd isotopes, 104Pd and 108Pd, loaded to MCP9 polymers generated higher signal intensities compared to their counterparts paired with mDOTA, while 105Pd paired with either MCP9 versus mDOTA resulted in similar signal intensities (FIG. 4C). The addition of three Pd isotopes loaded on MCP9 to the seven Cd isotopes loaded on MCP9 extended the barcoding toolbox to 10 barcoding reagents for multiplexing.

To test the 10 barcoding reagents, samples were labeled individually with different Cd or Pd tags and 10 samples were pooled before acquisition to reduce technical variation, assess channel noise, and simulate a barcoding scheme that enabled assessment of barcode separation. For Cd-tagged antibodies, at least a log 10 difference between CD45⁺ and CD45⁻ populations was observed for each individual, Cd-tagged antibody. The signal-to-noise ratio was less favorable for the Pd-tagged CD45 antibodies (FIG. 4D, FIG. 3B). The biaxial plots in FIG. 3B depict 45 different dual combinations observed when 10 tags were used. Nevertheless, there was obvious segregation among pooled samples labeled with single tags in the high-dimensional space (FIG. 4E, FIG. 3C).

### Example 4—Enhancement of Live-Cell Barcoding Capability Through Combined Use of Pd and Cd Isotopes

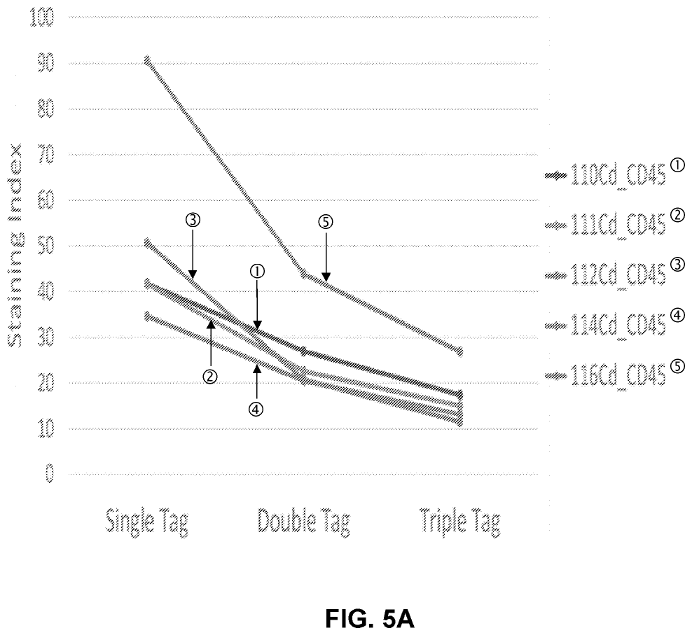

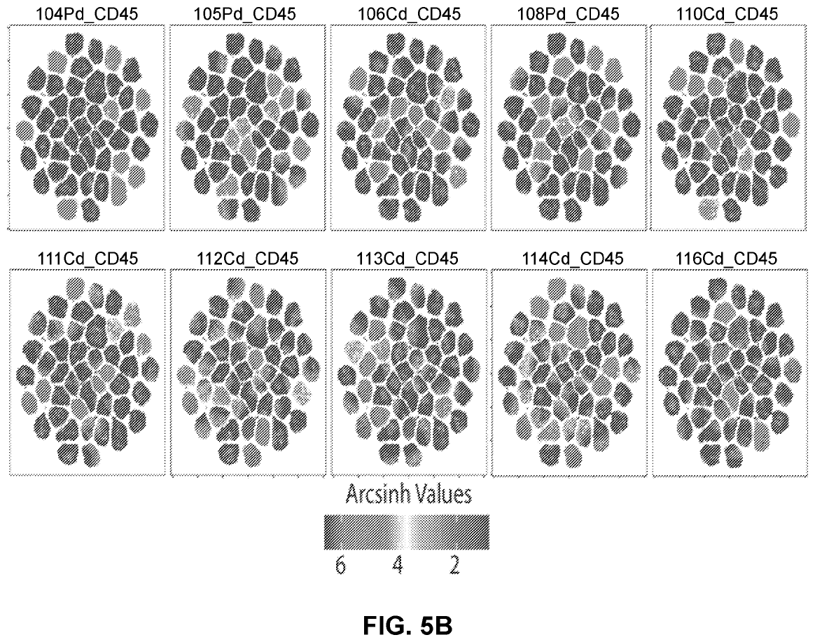

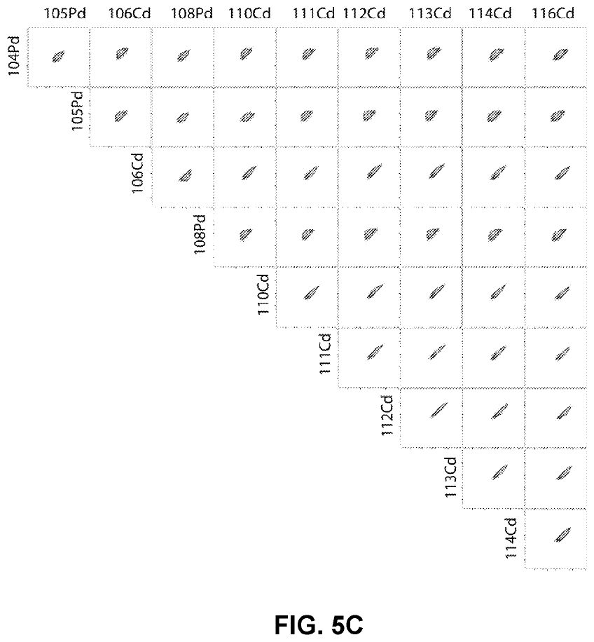

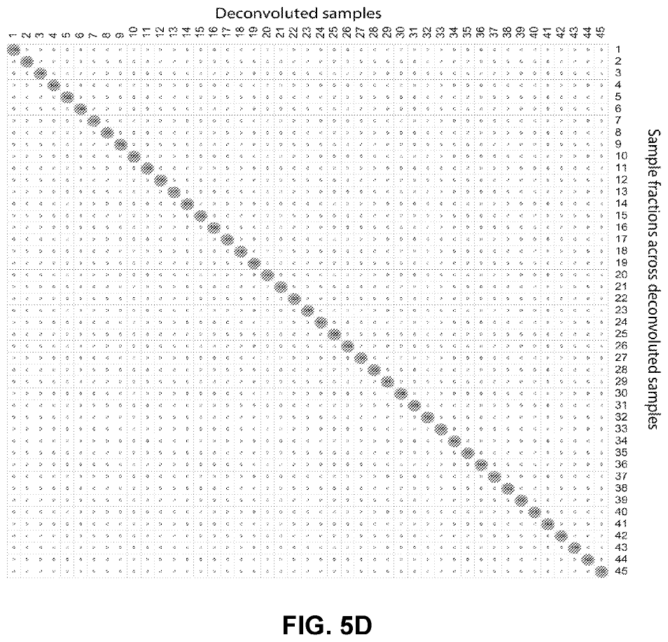

Ten different CD45-based barcoding reagents recognizing the same antigenic epitope were generated and various barcoding schemes were tested for multiplexing in CyTOF. Combined use of multiple tags to barcode a single sample decreased the signal intensity for any given single tag due to competition for the same antigenic epitope, which compromised signal resolution. Therefore, staining indices were assessed and compared by labeling samples with single, double, or triple combinations of five different Cd-tagged CD45 antibodies. Use of double or triple Cd-CD45 tags per individual sample was associated with a gradual decrease in the staining index (FIG. 5A).

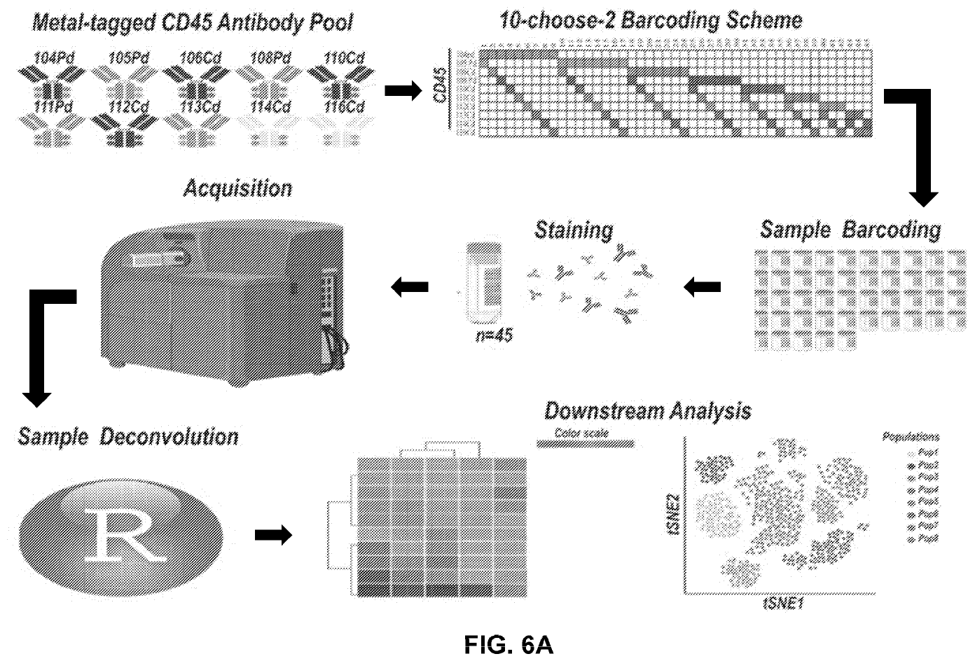

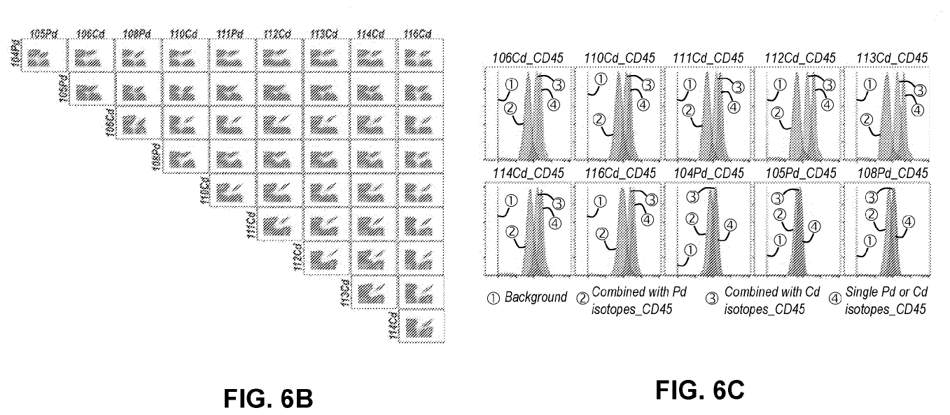

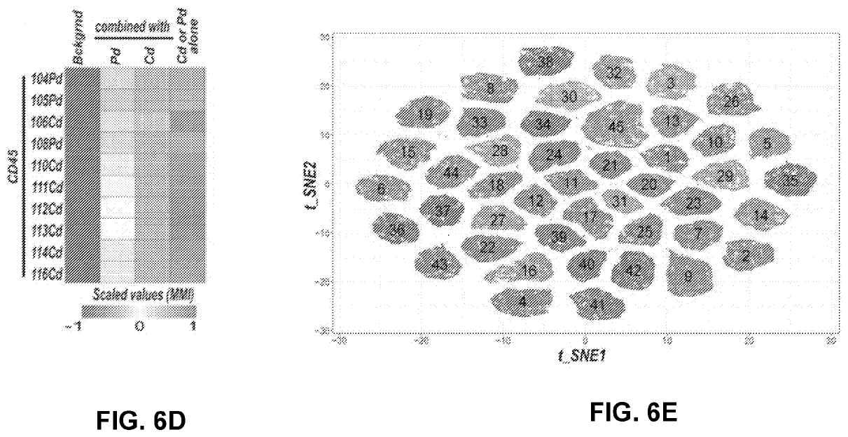

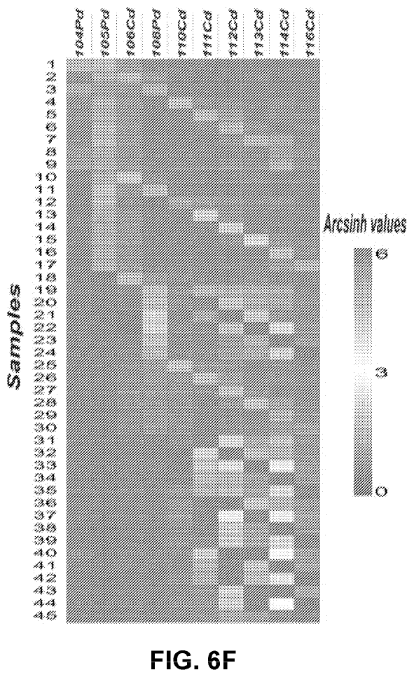

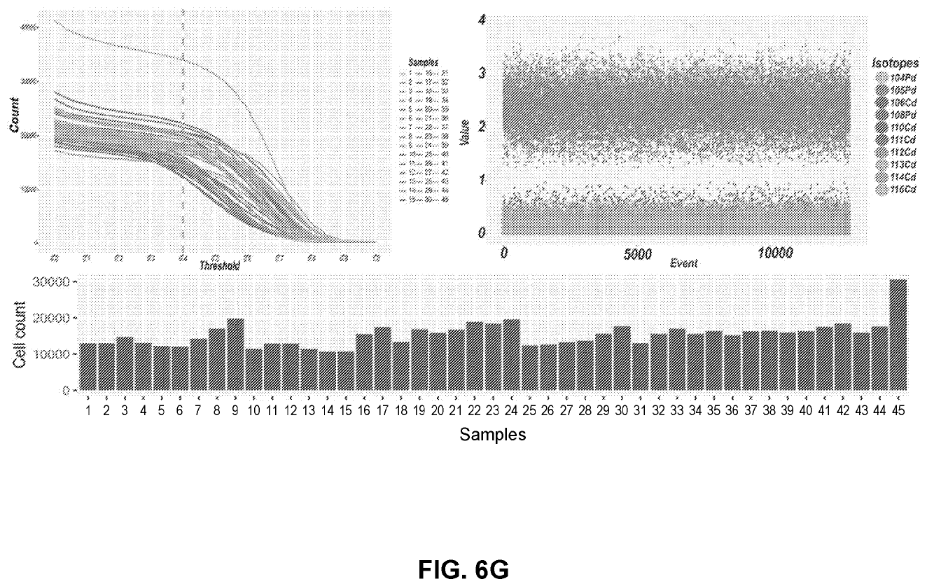

To maintain acceptable signal resolution, reduce antibody consumption and achieve higher yields after sample deconvolution two mass-tag cell barcodes (MCBs) were used per sample. This allowed a 10-choose-2 barcoding scheme and enabled barcoding of up to 45 experimental conditions. A schematic representation of a barcoding scheme (created with BioRender.com) shows that samples were individually barcoded with a unique MCB composed of two different Pd or Cd-tagged CD45 antibodies. The pooled samples were processed and acquired on a Helios™ CyTOF machine simultaneously. The samples were deconvoluted to their identity using a Premessa R package and the deconvoluted samples were then subjected to downstream analysis (FIG. 6A). To test the barcoding scheme, 45 dual antibody combinations were generated out of a pool of 10 Pd- and Cd-tagged CD45 antibodies and PBMCs from a single healthy donor were stained with the 45 MCBs and signal intensities of different dual combinations assessed. Separately labeled PBMCs were pooled together after labeling and run on a Helios™ CyTOF (FIG. 6B).

Combining any Cd-tagged CD45 antibodies with Pd-tagged CD45 antibodies resulted in marked signal reduction while relatively higher signal intensities were observed if the Cd-tagged CD45 antibodies were used together with CD45-tagged with different Cd isotopes (FIG. 6C). PBMCs were labeled with 45 MCBs, then pooled and acquired simultaneously. Positive events for each MCBs were selected and Cd or Pd positive events were gated out to calculate signal intensities in dual combinations with Pd and Cd isotopes, respectively. Conversely, combining Pd-tagged CD45 with either Pd- or Cd-tagged CD45 resulted in modest signal shifts (FIGS. 6C and 6D). However, despite a reduction in signal intensities with dual staining, each barcoded sample was distinctly represented when assigned barcoding tags were assessed using biaxial plots (FIG. 6B).

To assess the separation among all barcoded samples on a high dimensional plane, two-dimensional t-SNE (t-distributed stochastic neighbor embedding) maps were generated and PhenoGraph was used to partition the barcoded dataset into subpopulations. Barcoded samples were situated apart from each other and PhenoGraph identified 45 distinct subpopulations (FIG. 6E). Each sample exhibited a staining profile compatible with the assigned barcodes (FIG. 6F, FIG. 5B).

Having established that this extended barcoding scheme using Pd- and Cd-tagged CD45 antibodies enabled pooling of 45 samples with convincingly distinct barcode separation on a high-dimensional plane, samples were deconvoluted to their initial identity using Premessa R-package (FIG. 6G). As expected, deconvoluted samples displayed staining profiles compatible with their assigned barcodes (FIG. 5C). Ninety-three percent yield was achieved with applied filters (minimum barcode separation: 0.4, and Mahalanobis distance: 30).

TSNE and PhenoGraph analyses were performed on 45 de-barcoded samples to assess sample purity. The samples were examined to determine if they contained any cells from the bulk samples to assess purity. It was observed that each sample was composed of cells having unique assigned barcodes (FIG. 5D). Thus, the live-cell barcoding platform offered an attractive and feasible approach that could significantly contribute to the field of CyTOF by significantly augmenting sample throughput.

### Example 5—Use of Cd and Pd Labeled Antibodies to Analyze PBMCs of Healthy Individuals

To evaluate the utility of live-cell barcoding using this novel, extended CD45-based approach, frozen PBMCs were isolated from two healthy donors and other antibodies against abundantly expressed antigens were utilized for live-cell barcoding. B2M and CD298 antibodies were tagged with Cd and Pd isotopes and used in analyzing PBMCs.

PBMCs from two healthy donors were labeled with two different barcoding tags, 106Cd-B2M and 108Pd-B2M, and the samples were pooled. As a proof-of-concept, dual combinations of five different barcoding reagents that allowed barcoding for 10 different conditions were used. Pooled PBMCs from the two healthy donors were split into 10 aliquots, and each labeled with a unique MCB. Barcoded samples were pooled and a subsequently stained with a T-cell focused analyte panel (Table 1-category surface/intracellular/other).

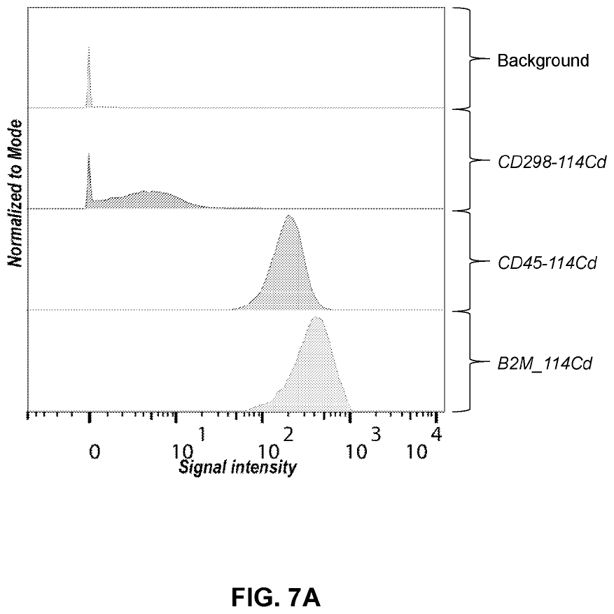

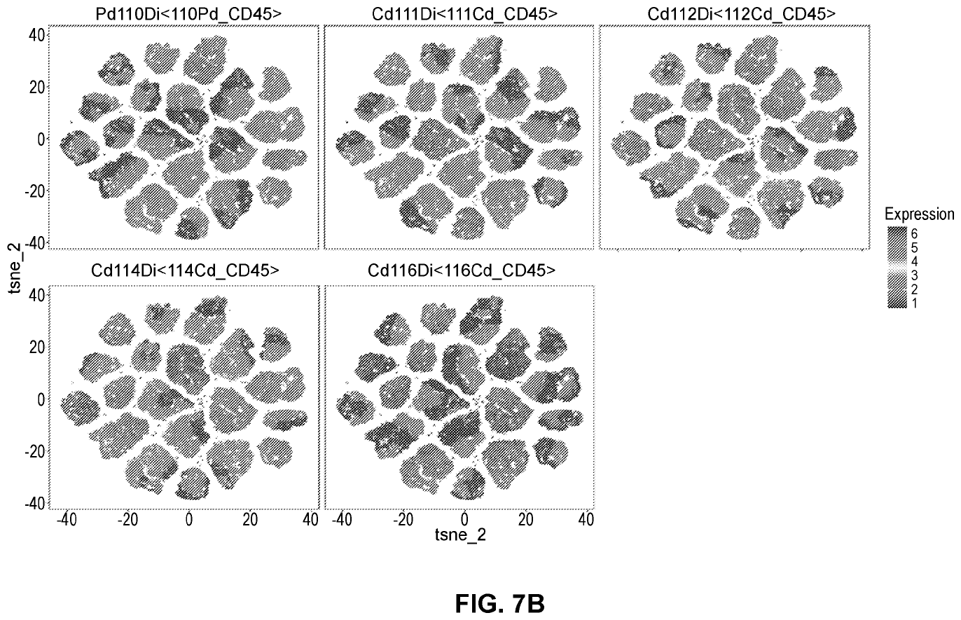

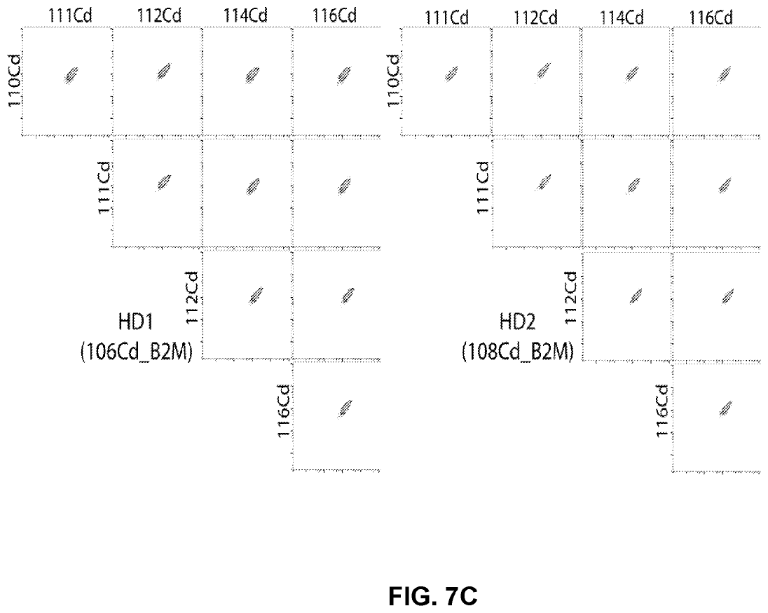

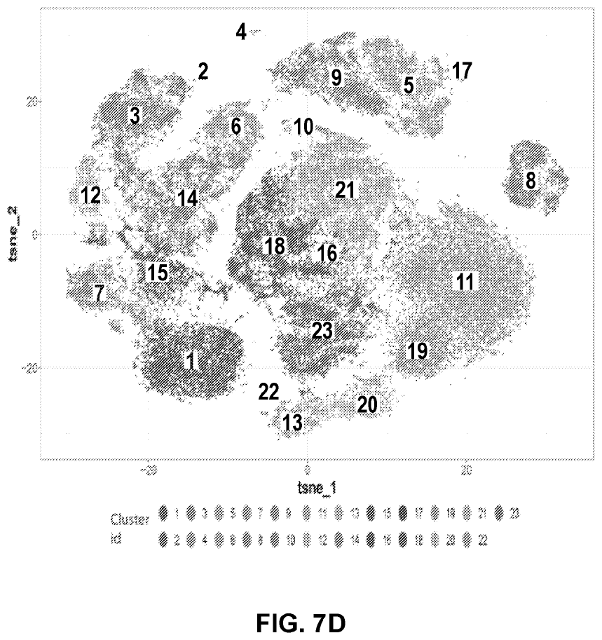

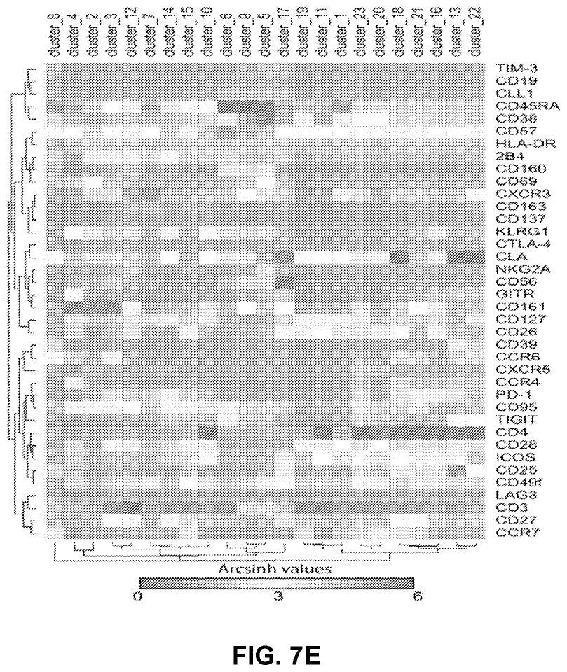

Antigen density of B2M on PBMCs was found to be comparable to CD45 while CD298 was less abundantly expressed (FIG. 7A).

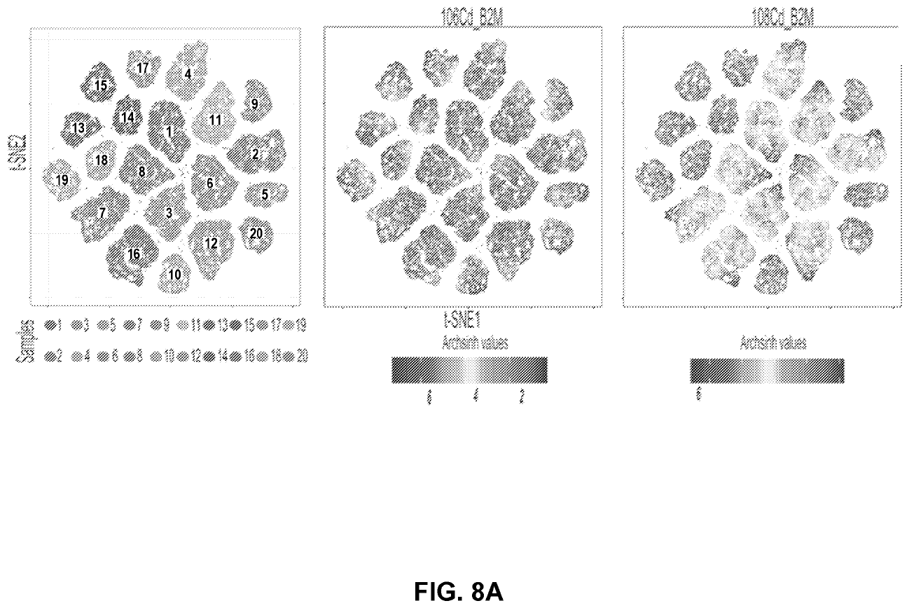

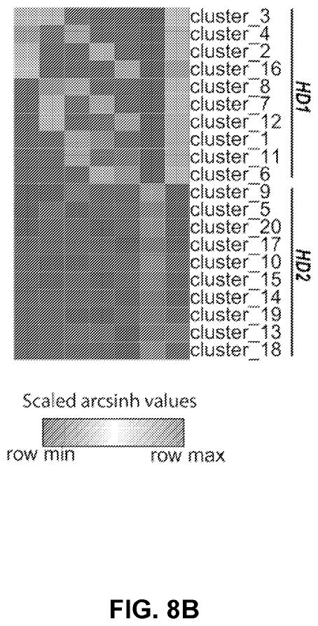

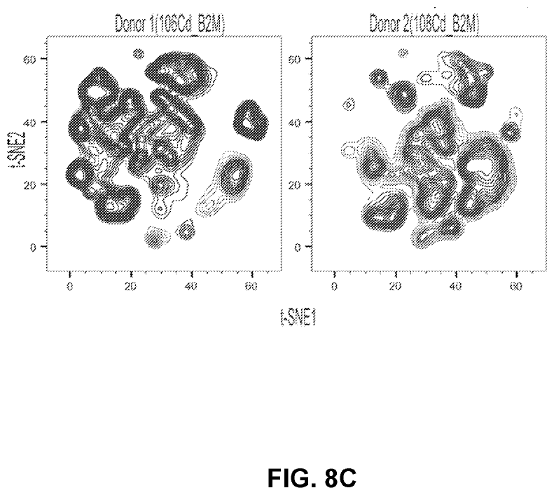

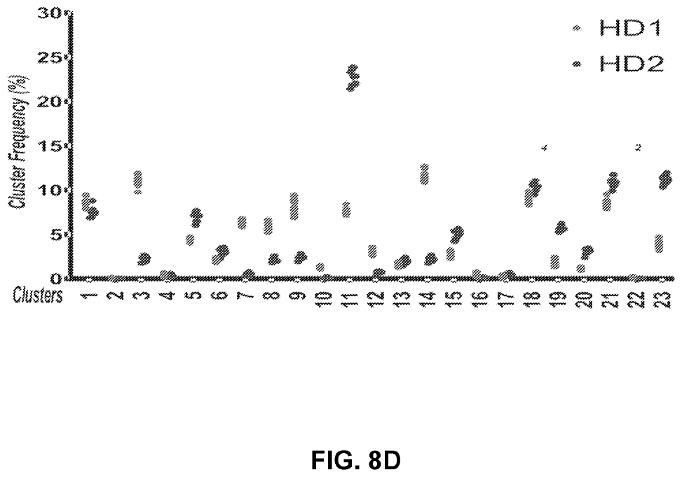

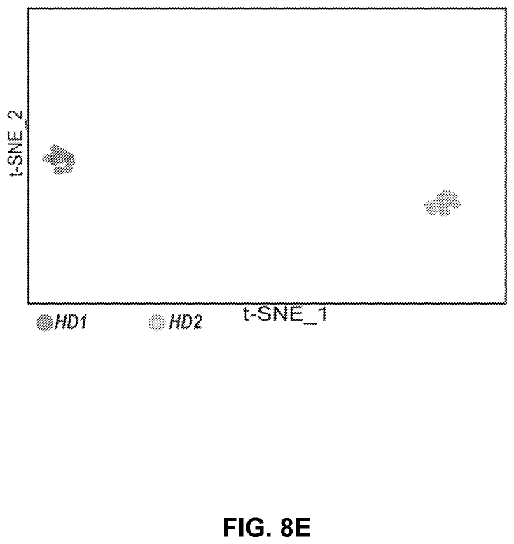

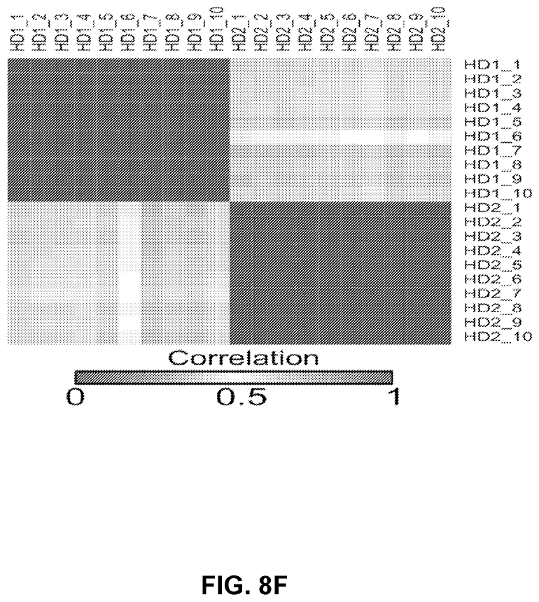

Dimension reduction and clustering using barcoding parameters identified 20 clusters (FIG. 8A), each having a phenotypic profile matching their assigned barcodes (FIG. 8B, FIG. 7B). FlowSOM was used to identify the clusters in the dataset. Five antibodies (110Cd, 111Cd, 112Cd, 114Cd, and 116Cd tagged to CD45) were used to generate 10 MCBs utilizing a 5-choose-2 barcoding scheme. Pooled PBMC from two HDs were subjected to a second round of barcoding using 10 MCBs. t-SNE maps were grayscaled for 106Cd_B2M (middle panel, HD1) and 108Pd_B2M (left panel, HD2) (FIG. 8A).

Premessa algorithm was used to assign the barcoded cells to their initial identity (FIG. 7C). T-SNE maps were generated using all deconvoluted samples and subpopulations in the dataset were identified using PhenoGraph (FIG. 7D). In this simulative approach, it was assumed that samples from the same donor would have the same cluster composition. Twenty-three distinct clusters were identified from 20 samples (2750 cells/sample) from the two healthy donors and their phenotypic profiles was evaluated. PhenoGraph revealed the major immune subsets observed in these two healthy donors (FIGS. 7D and 7E). Immune cluster composition differed between the two heathy donors (FIG. 8C). Deconvoluted samples (n=20) were subjected to the t-SNE algorithm using all parameters except barcoding tags for dimension reduction. Samples from the same donor had significantly similar cluster composition (FIG. 8D) and there was significant similarity among subsamples from the same donor with regards to cluster frequencies (FIGS. 8E and 8F), reflecting that samples were successfully allocated back to their original identity using this extended CD45-based barcoding approach and subsequent sample deconvolution. The approach proved the utility of the platform for multiplexing CyTOF and suggested that the platform can significantly streamline CyTOF analysis. The findings demonstrated the utility of this novel live-cell barcoding approach using a pool of MCP9-loaded Pd and Cd-tagged CD45 antibodies. The new approach substantially extended previous barcoding capabilities, enabled barcoding of significant number of samples, augmented throughput, minimized procedural variations, and enhanced data quality.

### Discussion of the Results of Examples 1-5

CyTOF allows simultaneous interrogation of a wide array of cellular features including cell surface profiles, cell cycle, proliferation, apoptosis, metabolism, phosphoproteins, cytokine production, transcription factors and multiplexed RNA profiles at single-cell resolution. Antibody staining variance, pipetting errors, detector drift between different samples on the same day and day-to-day variability of instrument performance are some of the sources of technical variation. Sample multiplexing reduces tube-to-tube variation, limits sample carry-over, decreases antibody consumption and enhances throughput.

For intracellular barcoding interrogating signaling pathways where a limited range of phenotypic profiling is sufficient to define dominant phenotypes, Pd isotopes can be used in fixed cells as they do not impinge on commonly used lanthanide tags.

However, for the assessment of surface phenotypes, fixed and permeabilized cells most likely do not reflect actual phenotypic profile. Fixation/permeabilization alters surface antigens by introducing conformational changes of the proteins and irreversibly modifying antigenic epitopes recognized by antibodies. Further, some surface antigens are also localized within intracellular granules and permeabilization quantifies these intracellular signals and, thus, cannot correctly assess surface signals.

To meet the growing demand for sample multiplexing while preserving surface epitopes, the current application provides innovative surface barcoding approaches. Metals other than lanthanides including Pd, platinum, tellurium, indium and yttrium were explored in the Examples described above for live-cell barcoding. Bifunctional chelating agents, ITCBE and mDOTA, were used to capture 6 Pd isotopes and generate MCBs for live-cell barcoding. However, the main limitation with Pd-based barcoding is that Pd isotopes elicit weaker signals. Therefore, 5 Pd isotopes were combined with other low sensitivity metals, such as indium isotopes, for sample multiplexing.

The weaker signals associated with Pd isotopes were partly related to the use of monomer chelators capable of capturing a limited number of metals compared to polymeric chelators. Use of MCP9 polymers specifically to chelate Cd isotopes improved signal intensities of this low sensitivity metal. Cd metals are situated at the lower end of the sensitivity spectrum and are traditionally paired only with abundant antigens, such as CD45. However, the use of MCP9 polymers described herein to capture higher numbers of metal isotopes increased signal intensities. In some examples, MCP9 was loaded with different Cd and Pd isotopes and paired with CD45, which is ubiquitously expressed on leukocytes although with varied expression across different subsets, to generate unique MCBs.

The live-cell barcoding scheme described in the Examples 1-5 included 10 mass tags, seven Cd and three Pd, and enabled to effectively pool up to 45 samples using a 10-choose-2 scheme. This significantly expanded the number of samples that could be barcoded using a live-cell barcoding approach in comparison to previous studies utilizing live-cell barcoding approaches. The approach was further extended to generate barcoding reagents by pairing MCP9/Cd isotopes with ubiquitously expressed markers, including B2M and CD298.

Seven Cd isotopes were loaded to MCP9 polymer and conjugated to CD45 antibodies. Having two overlapping isotopes with Pd (106Pd and 110Pd) enabled a comparison of signal intensities and metal content of similar mass weight, Cd-loaded MCP9 polymer versus Pd-loaded mDOTA monomer tagged with CD45 antibodies. The antibody capture bead approach demonstrated that the number of metals on MCP9 polymer was significantly higher compared to Pd loaded to mDOTA polymers (FIG. 1B). Thus, the availability of Cd/MCP9 for antibody tagging not only enriched the barcoding toolbox but also provided a means to generate superior signal intensities using low-sensitivity metals. Using three MCBs per sample out of seven enabled multiplexing of up to 35 samples. However, using three MCBs per sample was associated with lower staining index (FIG. 5A) and could possibly compromise total yield after sample deconvolution. Therefore, a two-tag per-sample approach was adopted to maintain favorable signal resolution and three Pd-tagged antibodies (104Pd, 105Pd, and 108Pd) were incorporated into the Cd-based barcoding scheme, allowing the use of 10 unique mass tags.

It was demonstrated for the first time that MCP9 can capture bivalent Pd metals. This approach offered great advantages over previously described antibody conjugation approaches using Pd isotopes loaded either to ITCBE or mDOTA. First, ITCBE is not readily dissolvable in “conjugation-friendly” buffers. Further, the chelator mDOTA readily dissolves in H₂O, but aqueous mDOTA is not stable. On the other hand, as demonstrated in the Examples provided herein, loading on MCP9 polymers significantly simplified and shortened antibody tagging with Pd isotopes and generated favorable signal intensities, albeit lower than Cd-tagged antibodies. The results indicated that MCP9 may have higher affinity for Cd than Pd isotopes. To load Pd metals to MCP9, Pd isotopes (104Pd, 105Pd, and 108Pd) already dissolved in 5 N HCl (hydrochloric acid) were used. Optimal pH is essential for metal chelation and Pd(NO3)2 is readily soluble in 100 mM 4-(2-hydroxyethyl)-1-piperazineethanesulfonic acid. Use of enriched Pd isotopes dissolved in 4-(2-hydroxyethyl)-1-piperazineethanesulfonic acid or H₂O to load onto MCP polymer can further improve chelation and augment signal intensities for Pd-MCP9 tagged CD45 antibodies.

In summary, a novel live-cell barcoding scheme based on combined use of Cd and Pd isotopes paired with CD45 was used to facilitate the pooling of a significantly higher number of samples with superior signal-to-noise ratios. This approach can be further tailored either through incorporating more mass tags residing at the lower end of the sensitivity spectrum to extend barcoding depth or using a certain of number MCBs to barcode a relatively lower number of samples while using the remaining isotopes to assess more analytes, as described herein.

Furthermore, since sample preparation and acquisition in CyTOF leads, in general, to significant cell loss requiring at least a million cells is starting material, The live-cell barcoding platform provided herein allows sample pooling, processing and simultaneous acquisition and enables interrogation of samples with very low cell counts, such as, e.g., samples of tumor cells and their microenvironment in patients with hypocellular bone marrows after treatment of hematological malignancies. Mapping the recovering immune system and residual tumor cells, termed minimal residual disease or MRD, is challenging due to therapy-related excessively low cell counts. Using the barcoding platform provided herein can address this challenge and provide significant benefits to clinical research and patient care.

### Example 6—Use of Y, Rh, Ru, Sn, La, Hg and Pb Labeled Antibodies to Analyze OCI-AML3 Cells

89Y was loaded on MCP9 and X8 polymers respectively. Detection sensitivity for 89Y was low when X8 polymers were used and use of MCP9-loaded CD45 resulted in improved signal intensities. X8 polymers do not chelate 103Rh at detectable ranges while MCP9 were capable of chelating 103Rh metals. 103Rh and 89Y were integrated in Cd and Pd based barcoding schemes to increase the number of experimental conditions that could be barcoded and pooled. MCP9 polymers are also able to capture other divalent metals including Sn, Pb and Hg. The availability of increased numbers of metal isotypes were used to assess a higher number of analytes by pairing isotope loaded MCP9 with different antibodies of interest. This approach significantly expanded the capabilities using CyTOF to achieve an in-depth profiling of any biological system of interest. X8 polymers can chelate 139La but metal-antibody conjugates were not stable. However, when loaded on MCP9 polymers metal tagged antibodies were stable and 139La tagged antibodies were incorporated in a CyTOF panel designed to assess distinct cellular features. Ruthenium isotopes were loaded on X8 polymers and did not precipitate if dissolved in at least 3% nitric acid solution. Ruthenium-tagged antibodies were used for sample barcoding alone or together with other barcoding reagents including 89Y, 103Rh, Pd and Cd isotopes.

Peripheral blood mononuclear cells, cell lines, and antibody capture beads were stained with metal tags generated as described in Example 1 to assess conjugation efficacy and ascertain that the conjugation process did not compromise antibody integrity. The staining of the antibody capture beads, mononuclear cell, and acute myeloid leukemia cell lines with novel-metal tagged antibodies elicited signal intensities with desirable signal-to-noise ratios, indicating that the X8 and MCP9 polymers were able to chelate and capture corresponding metals and metal-tagging of antibodies, and it did not alter or compromise the antigen-binding function.

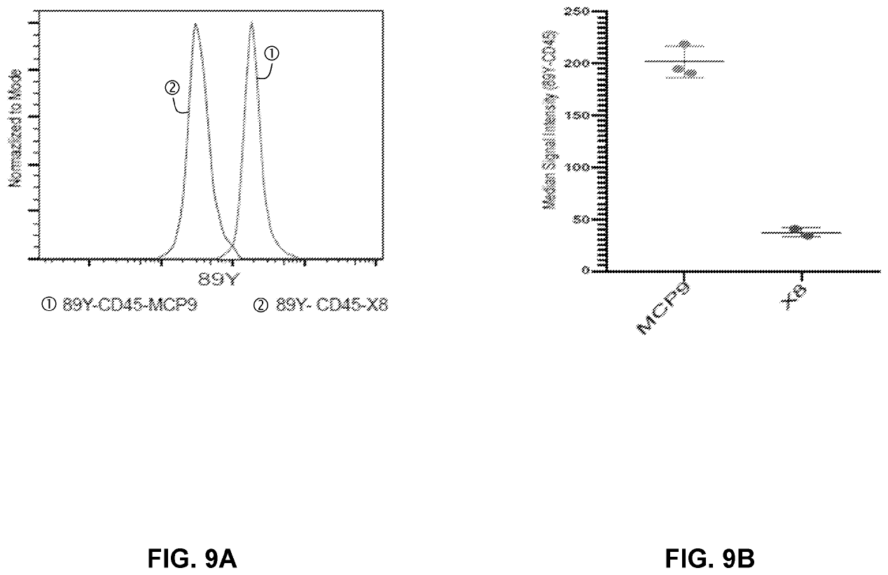

89Y loaded onto either X8 or MCP9 polymer, conjugated to CD45 antibodies and anti-mouse Ig kappa antibody capture beads demonstrated higher signal intensities for MCP9-loaded 89Y compared to X8-loaded 89Y (FIGS. 9A and 9B).

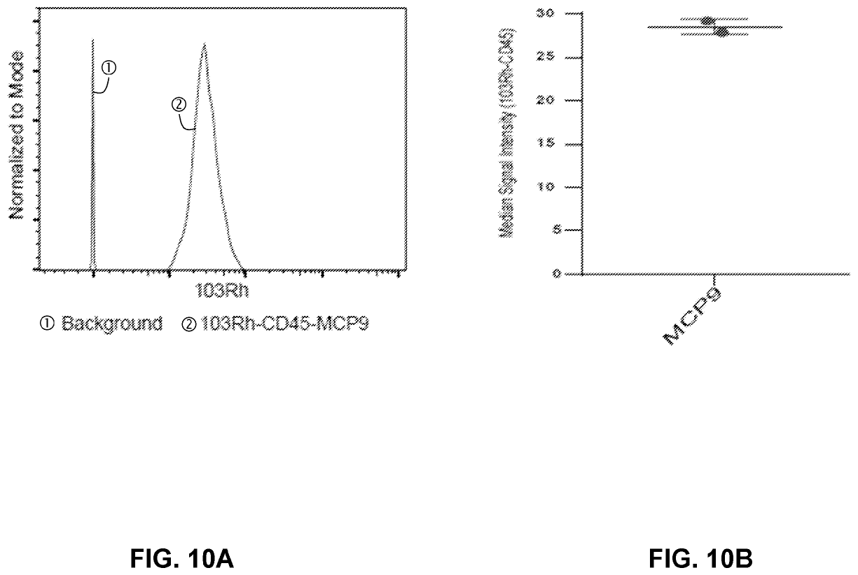

Rhodium chloride hydrate was dissolved in water, loaded onto MCP9 polymer and conjugated to CD45 antibodies and anti-mouse Ig kappa antibody capture beads. Histograms showed high signal intensities for MCP9-loaded 103Rh (FIGS. 10A and 10B).

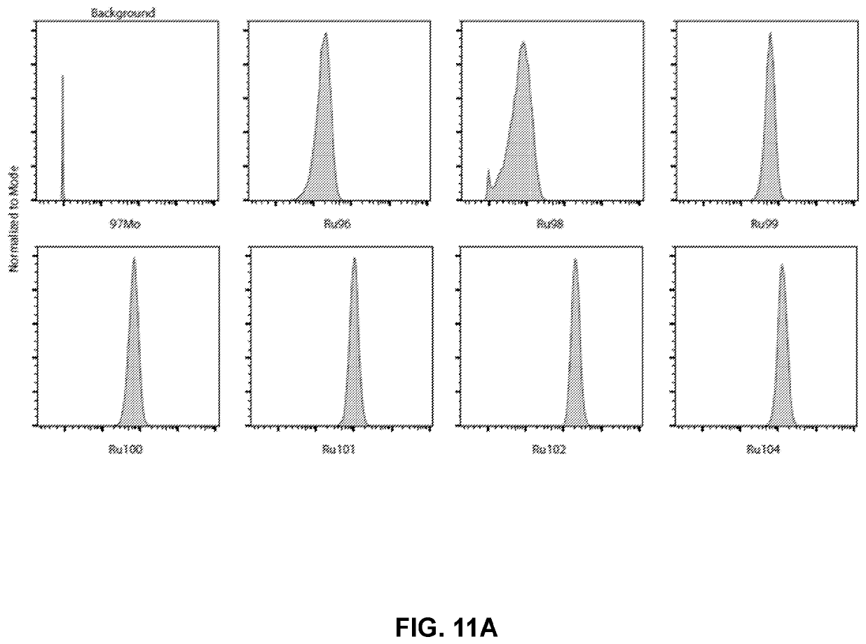

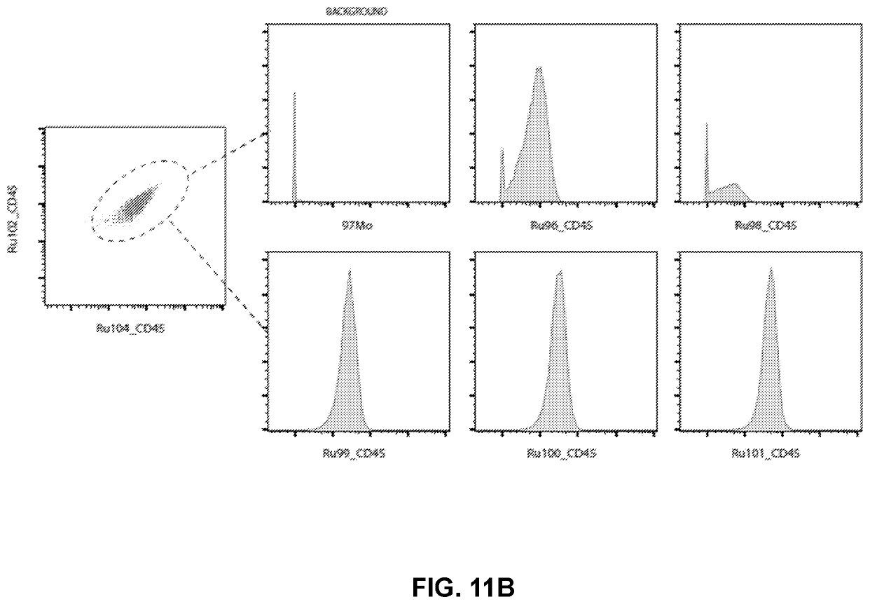

X8 polymer was used to chelate Ruthenium (Ru) isotopes. Ruthenium salt was dissolved in 3% nitric acid and loaded onto X8 polymer. CD45 antibody tagged with X8 polymer loaded with Ru isotopes were used to label antibody capture beads (FIG. 11A) and OCI-AML3 cell line (FIG. 11B). Signal intensities varied across different isotopes and reflected the natural abundance of isotopes in RuCl₃·xH₂O used for antibody conjugation. 97 Molybdenum (97Mo) was used as a negative control.

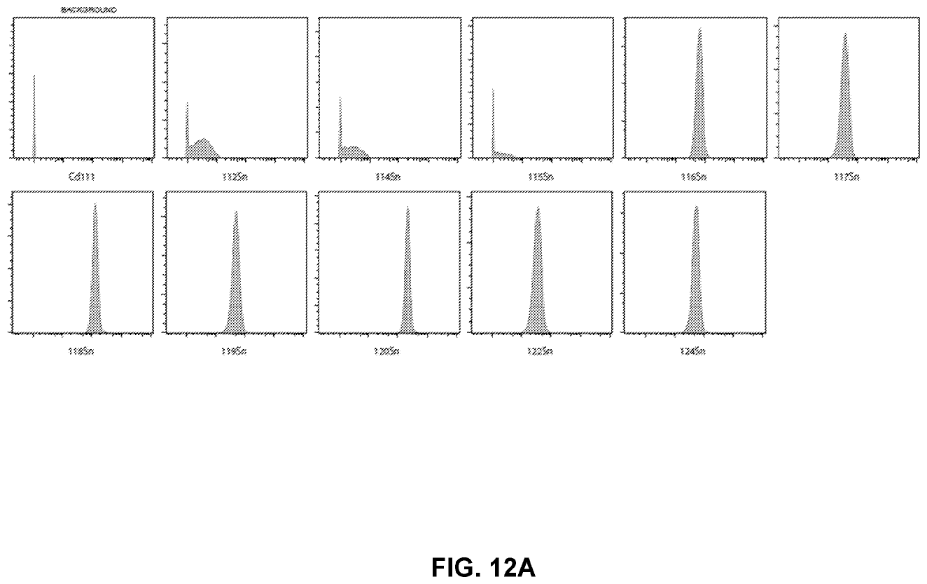

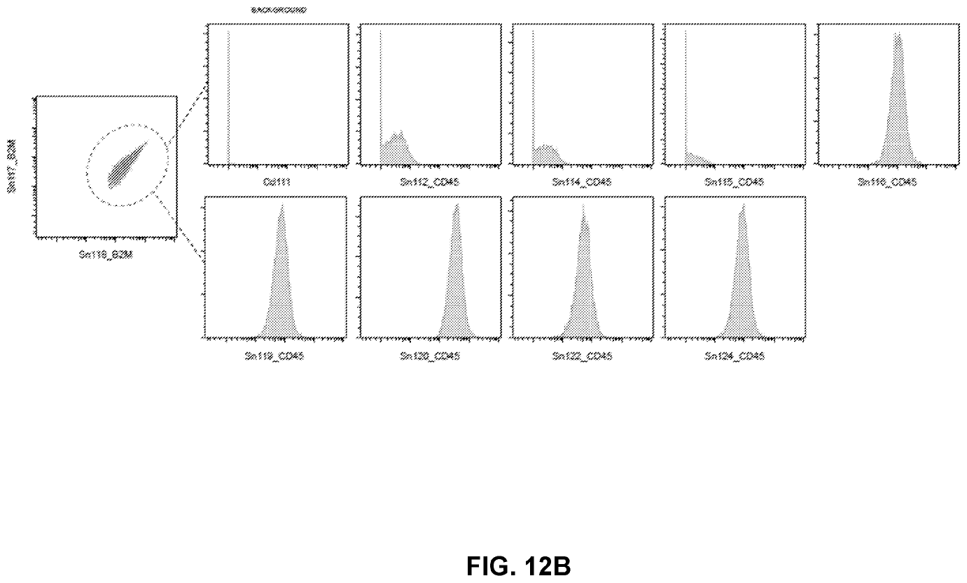

Tin chloride hydrate was dissolved in 5% nitric acid and loaded to MCP9 polymer. Seven Sn isotopes conjugated to CD45 antibodies showed high signal intensities when conjugated to capture beads (FIG. 12A) and OCI-AML3 cells (FIG. 12B). Signal intensities for 111Cd was used as a measure of background signal.

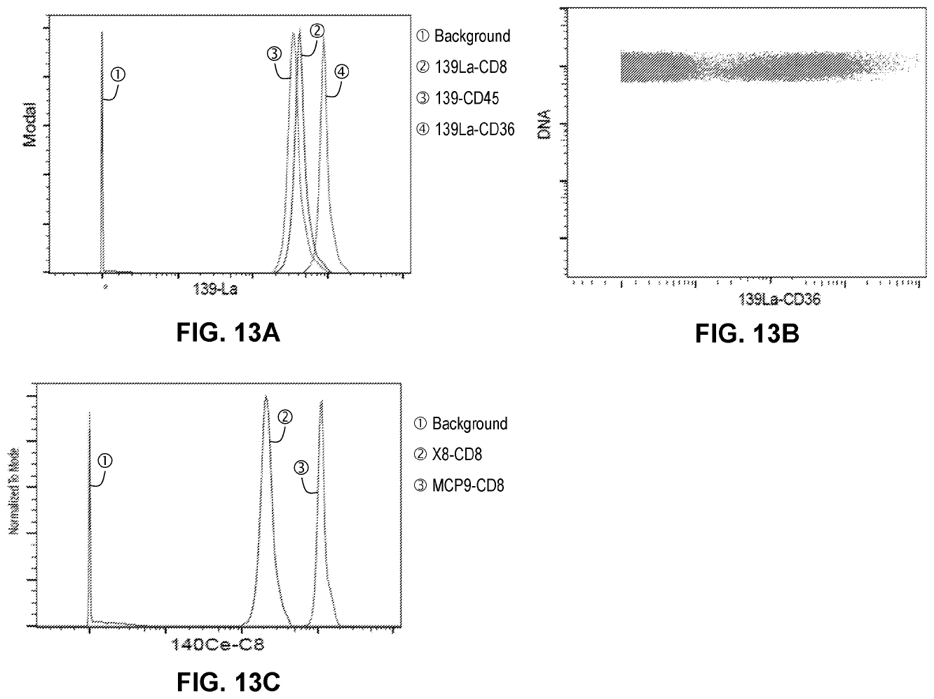

Lanthanum nitrate hydrate salt was dissolved in water and loaded onto MCP9 polymer. 139La-X8 conjugates were not stable while 139La-MCP9 conjugates were stable and CD36, CD45 and CD8 antibodies tagged with 139La-X8 elicited high signal intensities (FIG. 13A). OCI-AML3 cells and peripheral blood mononuclear cells from a healthy donor were mixed, stained with 139La-CD36 antibody and showed high 139La signal intensities (FIG. 13B). To compare the metal content captured by X8 and MCP9 polymers 140Ce (cerium) was loaded onto either X8 and MCP9 polymers and tagged to CD8 antibody. Signal intensities of 140Ce chelated by MCP9 polymer were higher than signal intensities of 140Ce chelated by X8 polymer (FIG. 13C).

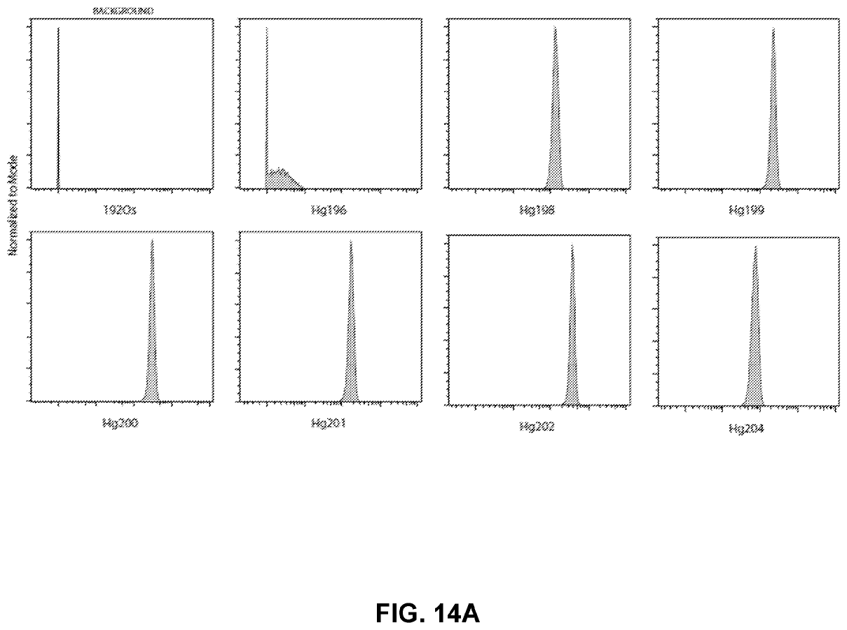

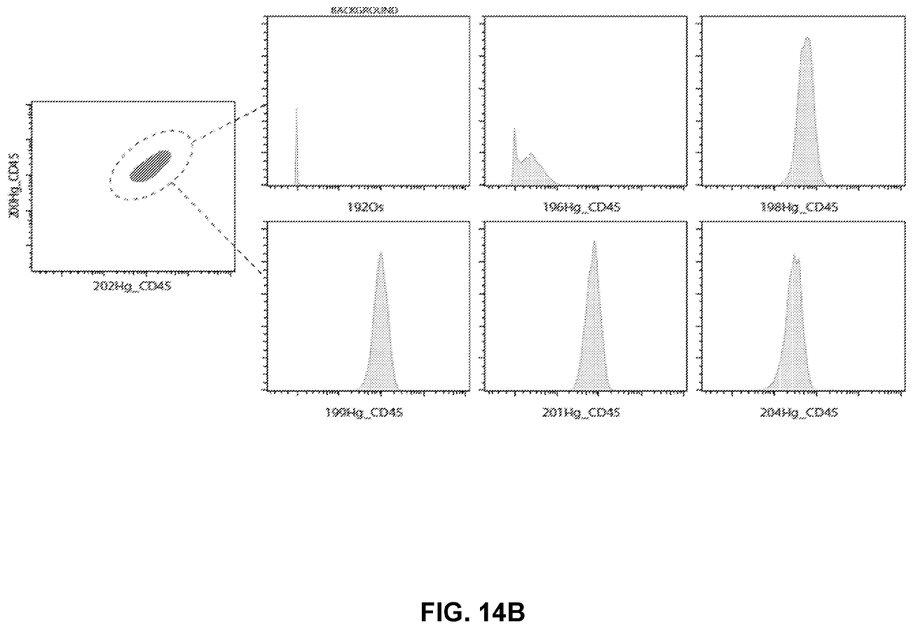

Mercury nitrate hydrate was dissolved in 5% nitric acid and loaded to MCP9 polymer. Seven Hg isotopes showed high signal intensities on beads (FIG. 14A) and OCI-AML3 cells (FIG. 14B). 192Os was used as negative control.

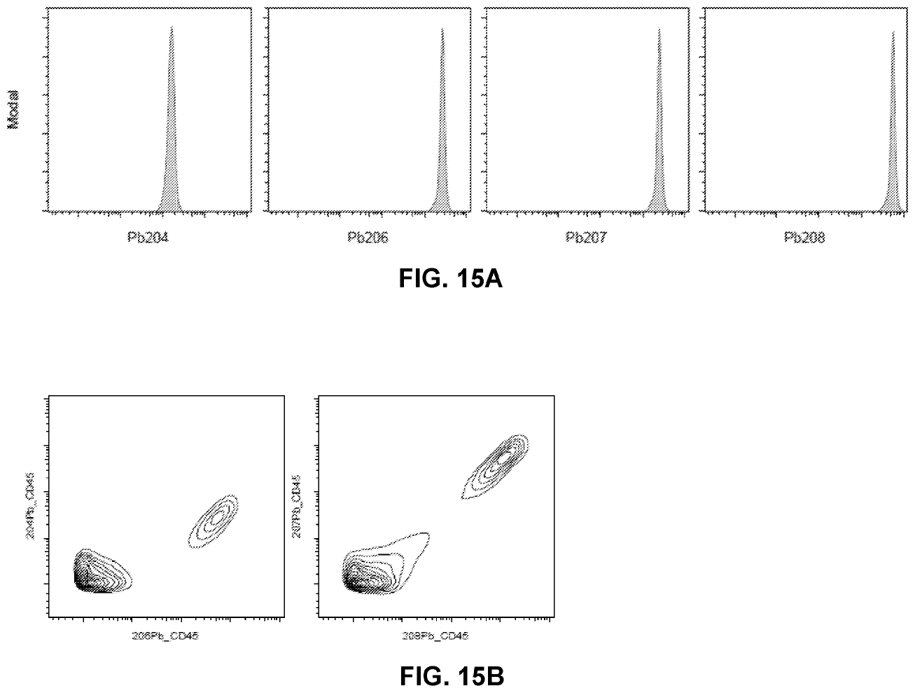

Lead nitrate was dissolved and loaded onto MCP9 polymer. Lead-MCP9 conjugate was tagged to CD45 and CD8 antibodies. Antibody capture beads were conjugated with 0.5 ug of Lead-CD8 antibody. Stained beads showed high signal intensities for 4 Pb isotopes (FIG. 15A). OCI-AML3 cells were stained with Pb-tagged CD45 antibody. Before acquisition, stained cells were mixed with unstained cells and acquired on CyTOF. Biaxial FACS plots show good separation between stained and unstained cells and high expression levels of CD45 on OCI-AML3 cells stained with 4 Pb isotope-tagged CD45 antibodies (FIG. 15 B).

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A population of metal tags, wherein each metal tag comprises:
a. a polymer comprising at least one metal-chelating group; and
b. an isotope;
wherein the polymer is selected from X8 polymer and metal-chelating polymer 9 (MCP9); the isotope has an atomic mass between about 89 and about 208; and at least one metal tag comprises an isotope selected from 204Pb, 206Pb, 207Pb, and 208Pb.

2. The population of metal tags of claim 1, wherein the isotope is selected from yttrium, ruthenium, rhodium, tin, lanthanum, mercury, rhenium, and combinations thereof.

3. The population of metal tags of claim 2, wherein the population of metal tags comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

4. The population of metal tags of claim 3, wherein the population of metal tags comprises at least four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

5. The population of metal tags of claim 3 or 4, wherein the population of metal tags comprises at least six isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

6. The population of metal tags of any one of claims 3-5, wherein the population of metal tags comprises at least eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

7. The population of metal tags of any one of claims 3-6, wherein the population of metal tags comprises at least ten isotopes selected from 89Y, 103Rh, 112Sn, 114Sn, 115Sn, 116Sn, 117Sn, 118Sn, 119Sn, 120Sn, 122Sn, 124Sn, 139La, and 138La.

8. The population of metal tags of any one of claims 3-7, wherein the population of metal tags comprises at least twelve isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

9. The population of metal tags of any one of claims 3-8, wherein the population of metal tags comprises at least fourteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

10. The population of metal tags of any one of claims 3-9, wherein the population of metal tags comprises at least sixteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

11. The population of metal tags of any one of claims 3-10, wherein the population of metal tags comprises at least eighteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

12. The population of metal tags of any one of claims 3-11, wherein the population of metal tags comprises at least twenty isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

13. The population of metal tags of any one of claims 3-12, wherein the population of metal tags comprises at least twenty-two isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

14. The population of metal tags of any one of claims 3-13, wherein the population of metal tags comprises at least twenty-four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

15. The population of metal tags of any one of claims 3-14, wherein the population of metal tags comprises at least twenty-six isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

16. The population of metal tags of any one of claims 3-15, wherein the population of metal tags comprises at least twenty-seven isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

17. The population of metal tags of any one of claims 3-16, wherein the population of metal tags comprises at least twenty-eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

18. The population of metal tags of any one of claims 3-17, wherein the population of metal tags comprises at least twenty-nine isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

19. The population of metal tags of any one of claims 3-18, wherein the population of metal tags comprises 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 185Re, and 187Re.

20. A population of metal tags, wherein each metal tag comprises:
a. a polymer comprising at least one metal-chelating group; and
b. an isotope;
wherein the polymer is selected from X8 polymer and metal-chelating polymer 9 (MCP9); the isotope has an atomic mass between about 89 and about 208; and at least one metal tag comprises 139La.

21. The population of metal tags of claim 20, wherein the isotope is selected from yttrium, ruthenium, rhodium, tin, mercury, lead, rhenium, and combinations thereof.

22. The population of metal tags of claim 21, wherein the population of metal tags comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

23. The population of metal tags of claim 22, wherein the population of metal tags comprises at least four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

24. The population of metal tags of claim 22 or 23, wherein the population of metal tags comprises at least eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

25. The population of metal tags of any one of claims 22-24, wherein the population of metal tags comprises at least twelve isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 196Hg, 198Hg, 199Hg, 200Hg, 201Hg, 202Hg, 204Hg, 204Pb, 206Pb, 207Pb, 208Pb, 185Re, and 187Re.

26. The population of metal tags of any one of claims 22-25, wherein the population of metal tags comprises at least sixteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

27. The population of metal tags of any one of claims 22-26, wherein the population of metal tags comprises at least twenty isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

28. The population of metal tags of any one of claims 22-27, wherein the population of metal tags comprises at least twenty-four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

29. The population of metal tags of any one of claims 22-28, wherein the population of metal tags comprises 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 138La, 196Hg, 198Hg, 199Hg, 200Hg, 201Hg, 202Hg, 204Hg, 204Pb, 206Pb, 207Pb, 208Pb, 185Re, and 187Re.

30. A population of metal tags, wherein each metal tag comprises:
a. a polymer comprising at least one metal-chelating group; and
b. an isotope;
wherein the polymer is selected from X8 polymer and metal-chelating polymer 9 (MCP9); the isotope has an atomic mass between about 89 and about 208; and at least one metal tag comprises an isotope selected from 185Re and 187Re.

31. The population of metal tags of claim 30, wherein the isotope is selected from yttrium, ruthenium, rhodium, tin, lanthanum, mercury, lead, and combinations thereof.

32. The population of metal tags of claim 31, wherein the population of metal tags comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn 122Sn, 124Sn, 139La, 138La, 196Hg, 198Hg, 199Hg, 200Hg, 201Hg, 202Hg, 204Hg, 204Pb, 206Pb, 207Pb, and 208Pb.

33. The population of metal tags of claim 32, wherein the population of metal tags comprises at least four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

34. The population of metal tags of claim 32 or 33, wherein the population of metal tags comprises at least eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

35. The population of metal tags of any one of claims 32-34, wherein the population of metal tags comprises at least twelve isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

36. The population of metal tags of any one of claims 32-35, wherein the population of metal tags comprises at least sixteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

37. The population of metal tags of any one of claims 32-36, wherein the population of metal tags comprises at least twenty isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

38. The population of metal tags of any one of claims 32-37, wherein the population of metal tags comprises at least twenty-four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

39. The population of metal tags of any one of claims 32-38, wherein the population of metal tags comprises 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 196Hg, 198Hg, 199Hg, 200Hg, 201Hg, 202Hg, 204Hg, 204Pb, 206Pb, 207Pb, and 208Pb.

40. A population of metal tags, wherein each metal tag comprises:
a. a polymer comprising at least one metal-chelating group; and
b. an isotope;
wherein the polymer is selected from X8 polymer and metal-chelating polymer 9 (MCP9); the isotope has an atomic mass between about 89 and about 208; and at least one metal tag comprises an isotope selected from 196Hg, 198Hg, 199Hg, 200Hg, 201Hg, 202Hg, and 204Hg.

41. The population of metal tags of claim 40, wherein the isotope is selected from yttrium, ruthenium, rhodium, tin, lanthanum, lead, rhenium, and combinations thereof.

42. The population of metal tags of claim 41, wherein the population of metal tags comprises one or more isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn 122Sn, 124Sn, 139La, 138La, 204Pb, 206Pb, 207Pb, 208Pb, 185Re, and 187Re.

43. The population of metal tags of claim 42, wherein the population of metal tags comprises at least four isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

44. The population of metal tags of claim 42 or 43, wherein the population of metal tags comprises at least six isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

45. The population of metal tags of any one of claims 42-44, wherein the population of metal tags comprises at least eight isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

46. The population of metal tags of any one of claims 42-45, wherein the population of metal tags comprises at least ten isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

47. The population of metal tags of any one of claims 42-46, wherein the population of metal tags comprises at least twelve isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

48. The population of metal tags of any one of claims 42-47, wherein the population of metal tags comprises at least thirteen isotopes selected from 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

49. The population of metal tags of any one of claims 42-48, wherein the population of metal tags comprises 89Y, 96Ru, 98Ru, 99Ru, 100Ru, 101Ru, 102Ru, 104Ru, 103Rh, 112Sn, 114Sn, 115Sn, 116Sn, 117Sn, 118Sn, 119Sn, 120Sn, 122Sn, 124Sn, 139La, 138La, 204Pb, 206Pb, 207Pb, 208Pb, 185Re, and 187Re.

50. A population of metal tags, wherein each metal tag comprises:
a. a polymer comprising at least one metal-chelating group; and
an isotope; b,
wherein the polymer is selected from X8 polymer and metal-chelating polymer 9 (MCP9); the isotope has an atomic mass between about 89 and about 208; and at least one metal tag comprises 103Rh.

51. The population of metal tags of claim 50, wherein the polymer is MCP9.

52. The population of metal tags of claim 51, wherein the isotope is selected from cadmium, palladium, indium, yttrium, and combinations thereof.

53. The population of metal tags of claim 52, wherein the population of metal tags comprises one or more isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In.

54. The population of metal tags of claim 53, wherein the population of metal tags comprises at least four isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In.

55. The population of metal tags of claim 53 or 54, wherein the population of metal tags comprises at least six isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In.

56. The population of metal tags of any one of claims 53-55, wherein the population of metal tags comprises at least eight isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In.

57. The population of metal tags of any one of claims 53-56, wherein the population of metal tags comprises at least ten isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In.

58. The population of metal tags of any one of claims 53-57, wherein the population of metal tags comprises at least twelve isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In.

59. The population of metal tags of any one of claims 53-58, wherein the population of metal tags comprises at least fourteen isotopes selected from 106Cd, 110Cd, 111Cd, 112Cd, 113Cd, 114Cd, 116Cd, 102Pd, 104Pd, 105Pd, 106Pd, 108Pd, 110Pd, 89Y, and 115In.

60. The population of metal tags of any one of claims 53-59, wherein the population of metal tags comprises 106Cd, 110Cd, 111Cd, 112Cd, 113Cd, 114Cd, 116Cd, 102Pd, 104Pd, 105Pd, 106Pd, 108Pd, 110Pd, 89Y, and 115In.

61. The population of metal tags of any one of claims 53-60, wherein the population of metal tags comprises 106Cd, 110Cd, 111Cd, 112Cd, 113Cd, 114Cd, 116Cd, 102Pd, 104Pd, 105Pd, 108Pd, 89Y, and 115In.

62. A population of metal tags that comprises at least one metal tag of the population of metal tags of claim 53 and at least one metal tag of the population of metal tags of claim 32.

63. A population of metal tags that comprises at least one metal tag of the population of metal tags of claim 53 and at least one metal tag of the population of metal tags of claim 42.

64. A population of metal tags that comprises at least one metal tag of the population of metal tags of claim 32 and at least one metal tag of the population of metal tags of claim 42.

65. A population of metal tags that comprises at least one metal tag of the population of metal tags of claim 53, at least one metal tag of the population of metal tags of claim 32, and at least one metal tag of the population of metal tags of claim 42.

66. The population of metal tags of claim 32, wherein the polymer is X8 polymer and the population of metal tags comprises one or more isotopes selected from 96Ru, 87Ru, 99Ru, 100Ru, 101Ru, 102Ru, and 104Ru.

67. The population of metal tags of claim 32, wherein the population of metal tags comprises isotopes 103Rh, 89Y, and 139La.

68. The population of metal tags of claim 67, further comprising metal tags that comprise one or more isotopes selected from 106Cd, 110Cd, 111Cd, 112Cd, 113Cd, 114Cd, 116Cd, 102Pd, 104Pd, 105Pd, 106Pd, 108Pd, 110Pd, and 115In.

69. The population of metal tags of claim 68, further comprising metal tags that comprise one or more isotopes selected from 96Ru, 87Ru, 99Ru, 100Ru, 101Ru, 102Ru, and 104Ru.

70. The population of metal tags of claim 69, further comprising metal tags that comprise one or more isotopes selected from 112Sn, 114Sn, 115Sn, 116Sn, 117Sn, 118Sn, 119Sn, 120Sn, 122Sn 122Sn, 124Sn, 138La, 196Hg, 198Hg, 199Hg, 200Hg, 201Hg, 202Hg, 204Hg, 185Re, 187Re 204Pb, 206Pb, 207Pb, and 208Pb.

71. A population of metal-tagged probes, wherein each metal-tagged probe comprises:
a. a metal tag of a population of metal tags according to any one of claims 1-61; and
b. a biomolecule;
wherein the metal tag is conjugated to the biomolecule.

72. The population of metal-tagged probes of claim 71, wherein the biomolecule is a protein.

73. The population of metal-tagged probes of claim 72, wherein the protein is an antibody.

74. The population of metal-tagged probes of any one of claims 71-73, wherein the protein and/or antibody has at least one sulfhydryl group and the metal tag is conjugated to the at least one sulfhydryl group.

75. The population of metal-tagged probes of claim 74, wherein the antibody is selected from an anti-CD45, anti-CD19, anti-CD3, anti-CD4, anti-CD8, anti-TNFα, anti-IFN-γ, anti-CD16, anti-CD33, anti-CD36, anti-phosphoSTAT5, anti-IL2, anti-CD28, anti-CD14, anti-T-bet, anti-CD66b, anti-B2M, anti-CD20, anti-HLA-DR, anti-CD14, anti-CD56, anti-CD294, anti-CD38, anti-CD11c, anti-CD57, anti-CD123, anti-TCRγδ, anti-CD161, anti-CCR7, anti-CD45RA, anti-CD27, anti-CD45RO, anti-CD25, anti-CCR5, anti-CCR4, anti-CXCR3, and anti-CCR6, anti-CD26, anti-NKG2A, anti-CD49F, anti-CD127, anti-CCR2, anti-TIGIT, anti-Ki-67, anti-TIM3, anti-2B4, anti-ICOS, anti-PD-1, anti-Eomes, anti-CD137, anti-CTLA-4, anti-LAG3, anti-Perforin, anti-CD160, anti-CD69, anti-CD39, anti-CD73, anti-KLRG1, anti-CD95, anti-GITR, anti-CLA. anti-CD298, anti-CD98, anti-G6PD, anti-GAPDH, anti-CD15S, anti-pS6, anti-pAKT, anti-MEK1/2, anti-pERK1/2, anti-phosphoSTAT3, anti-phosphoSTAT1, anti-phosphoSTAT4, anti-phosphoSTAT6, anti-p4EBP1, anti-GLUT1, anti-H3K27, anti-c-myc, anti-granzyme A, anti-granzyme B, anti-granzyme K, anti-granzyme M, anti-Helios, anti-RUNX3, anti-p53, anti-HIF1a, anti-p-NRF2, anti-Bcl-2, anti-Mcl-1, anti-Bcl-xL, anti-BIM, anti-Bax, anti-CD90, anti-CD96, anti-OX40, anti-MIP1a, anti-MIP1b, anti-IL4, anti-IL5, anti-IL17A, anti-IL6, anti-MCP1, anti-CD112, anti-CDD115, anti-CD34, anti-CD11b, anti-CD11a, anti-CD141, anti-CD105, anti-CD1a, anti-CD1c, anti-CD235a, anti-YTHDF2, anti-CD41a, anti-CD71, anti-Siglec7, anti-CD30, anti-CD61, anti-RANTES, anti-MCT-1, anti-MCT4, anti-CD80, anti-CD83, anti-CD86, anti-KIR3DL1, anti-KIR3DL2, anti-KIR2DL1, anti-NKG2C, anti-CD94, anti-NRF2, anti-CDT1, anti-pRB, anti-CyclinB1, anti-BrU, and combinations thereof.

76. The population of metal-tagged probes of any one of claims 71-75, wherein the metal-tagged probes are capable of detecting a cell target at a level of increased sensitivity compared to a metal-tagged probe that does not comprise a metal tag of the population of metal tags of claims 53, 32, and/or 42.

77. A method of generating a metal tag, the method comprising
a. providing an isotope salt;
b. dissolving the isotope salt in a solvent;
c. providing a polymer; and
d. loading the isotope onto the polymer to generate a metal tag;
wherein the isotope salt is selected from the group consisting of yttrium (III) chloride hydrate, yttrium (III) nitrate hydrate, rhodium (III) chloride hydrate, ruthenium (III) chloride hydrate, palladium (II) nitrate hydrate, cadmium (II) nitrate, indium (III) chloride, tin (II) chloride hydrate, mercury (II) nitrate hydrate, rhenium (III) chloride, lead (II) nitrate, and lanthanum (III) nitrate hydrate.

78. The method of claim 77, wherein the polymer is X8 or MCP9.

79. The method of claim 77 or 78, wherein the solvent is selected from water (pH about 6), hydrochloric acid, nitric acid, 4-(2-hydroxyethyl)-1-piperazineethanesulfonic acid, and combinations thereof.

80. The method of claim 79, wherein the isotope salt is yttrium (III) chloride hydrate and the solvent is water (pH about 6).

81. The method of claim 79, wherein the isotope salt is yttrium (III) chloride hydrate, the polymer is MCP9, and the solvent is water (pH about 6).

82. The method of claim 79, wherein the isotope salt is yttrium (III) nitrate hydrate and the solvent is water (pH about 6).

83. The method of claim 79, wherein the isotope salt is yttrium (III) nitrate hydrate, the polymer is MCP9, and the solvent is water (pH about 6) or L buffer from Fluidigm® (pH about 6).

84. The method of claim 79, wherein the isotope salt is ruthenium (III) chloride hydrate and the solvent is between about 3% nitric acid and about 5% nitric acid.

85. The method of claim 79, wherein the isotope salt is ruthenium (III) chloride hydrate, the polymer is X8, and the solvent is about 3% nitric acid.

86. The method of claim 79, wherein the ruthenium (III) chloride is loaded onto the X8 polymer in the presence of about 3% nitric acid.

87. The method of claim 79, wherein the isotope salt is rhodium (III) chloride hydrate, and the solvent is water (pH about 6).

88. The method of claim 79, wherein the isotope salt is rhodium (III) chloride hydrate, the polymer is MCP9, and the solvent is water (pH about 6) or L buffer from Fluidigm® (pH about 6).

89. The method of claim 79, wherein the isotope salt is palladium (II) nitrate hydrate and the solvent is water (pH about 6).

90. The method of claim 79, wherein the isotope salt is palladium (II) nitrate hydrate, the polymer is MCP9, and the solvent is water (pH about 6) or L buffer from Fluidigm® (pH about 6).

91. The method of claim 79, wherein the isotope salt is cadmium (II) nitrate and the solvent is L buffer from Fluidigm® (pH about 6).

92. The method of claim 79, wherein the isotope salt is cadmium (II) nitrate, the polymer is MCP9, and the solvent is L buffer from Fluidigm® (pH about 6).

93. The method of claim 79, wherein the isotope salt is tin (II) chloride hydrate and the solvent is between about 3% nitric acid and about 5% nitric acid.

94. The method of claim 79, wherein the isotope salt used in the method is tin (II) chloride hydrate, the polymer is MCP9, and the solvent is about 5% nitric acid.

95. The method of claim 79, wherein the isotope salt is indium (III) chloride and the solvent is water (pH about 6) or L buffer from Fluidigm® (pH about 6).

96. The method of claim 79, wherein the isotope salt is indium (III) chloride, the polymer is X8, and the solvent is water (pH about 6) or L buffer from Fluidigm® (pH about 6).

97. The method of claim 79, wherein the isotope salt is mercury (II) nitrate hydrate and the solvent is between about 3% nitric acid and about 5% nitric acid.

98. The method of claim 79, wherein the isotope salt is mercury (II) nitrate hydrate, the polymer is MCP9, and the solvent is about 5% nitric acid.

99. The method of claim 79, wherein the isotope salt is lead (II) nitrate and the solvent is water (pH about 6).

100. The method of claim 79, wherein the isotope salt is lead (II) nitrate, the polymer is MCP9, and the solvent is water (pH about 6) or L buffer from Fluidigm® (pH about 6).

101. The method of claim 79, wherein the isotope salt is rhenium (III) chloride and the solvent is between about 3% nitric acid and about 5% nitric acid.

102. The method of claim 79, wherein the isotope salt is rhenium (III) chloride, the polymer is X8, and the solvent is about 3% nitric acid.

103. The method of claim 79, wherein the isotope salt is lanthanum (III) nitrate hydrate, and the solvent is water (pH about 6).

104. The method of claim 79, wherein the isotope salt is lanthanum (III) nitrate hydrate, the polymer is MCP9, and the solvent is water (pH about 6) or L buffer from Fluidigm® (pH about 6).

105. A method of producing a metal-tagged antibody, the method comprising:
a. providing an antibody;
b. partially reducing the antibody to generate sulfhydryl groups;
c. providing a metal tag that is generated according to the method of any one of claims 77-104;
d. conjugating the metal tag to the partially reduced sulfhydryl group of the antibody to produce the metal-tagged antibody.

106. A kit comprising a population of metal tags according to any one of claims 1-70 and one or more reagents that facilitate conjugation of at least one antibody to a metal tag of the population of metal tags.

107. A kit comprising a population of metal-tagged probes according to any one of claims 71-76.

108. The kit of claim 106 or 107, further comprising a plurality of antibodies, wherein each antibody targets a different antigen and all antibodies comprise the same metal tag.

109. The kit of claim 106 or 107, further comprising a plurality of antibodies wherein each antibody targets a different antigen and some antibodies comprise the same metal tag and some antibodies comprise metal tags that are different from each other.

110. The kit of any one of claims 107-109 further comprising a calibration material comprising a known quantity of an isotope present in a metal tag of the population of metal tags and/or the population of metal-tagged probes.

111. The kit of claim 106, further comprising a plurality of reagents for labeling a plurality of samples, wherein each of the plurality of reagents comprises a distinct combination of metal tags.

112. The kit of claim 106, further comprising a plurality of antibodies,

113. The kit of claim 112, wherein each antibody comprises all metal tags of the population of metal tags.

114. The kit of claim 112, wherein each antibody comprises a distinct combination of metal tags of the population of metal tags.

115. The kit of any one of claims 106-114, further comprising a plurality of hermetically sealed containers.

116. The kit of claim 115, wherein each of the plurality of sample barcoding reagents is contained within different containers of the plurality of hermetically sealed containers.

117. A panel for elemental analysis comprising: a plurality of conjugated antibodies, wherein each of the plurality of conjugated antibodies is tagged with a distinct metal tag of any one of claims 1-70, wherein each distinct metal tag is distinguishable based on its isotopic composition, and wherein the plurality of conjugated antibodies is in a mixture.

118. The panel of claim 117, wherein the plurality of conjugated antibodies include two or more antibodies selected from anti-CD45, anti-CD19, anti-CD3, anti-CD4, anti-CD8, anti-TNFα, anti-IFN-γ, anti-CD16, anti-CD33, anti-CD36, anti-phosphoSTAT5, anti-IL2, anti-CD28, anti-CD14, anti-T-bet, anti-CD66b, anti-B2M, anti-CD20, anti-HLA-DR, anti-CD14, anti-CD56, anti-CD294, anti-CD38, anti-CD11c, anti-CD57, anti-CD123, anti-TCRγδ, anti-CD161, anti-CCR7, anti-CD45RA, anti-CD27, anti-CD45RO, anti-CD25, anti-CCR5, anti-CCR4, anti-CXCR3, and anti-CCR6, anti-CD26, anti-KG2A, anti-CD49F, anti-CD127, anti-CCR2, anti-TIGIT, anti-Ki-67, anti-TIM3, anti-2B4, anti-ICOS, anti-PD-1, anti-Eomes, anti-CD137, anti-CTLA-4, anti-LAG3, anti-Perforin, anti-CD160, anti-CD69, anti-CD39, anti-KLRG1, anti-CD95, anti-GITR, anti-CLA, anti-CD73, Anti-CD298, anti-CD98, anti-G6PD, anti-GAPDH, anti-CD15S, anti-pS6, anti-pAKT, anti-MEK1/2, anti-pERK1/2, anti-phosphoSTAT3, anti-phosphoSTAT1, anti-phosphoSTAT4, anti-phosphoSTAT6, anti-p4EBP1, anti-GLUT1, anti-H3K27, anti-c-myc, anti-granzyme A, anti-granzyme B, anti-granzyme K, anti-granzyme M, anti-Helios, anti-RUNX3, anti-p53, anti-HIF1a, anti-p-NRF2, anti-Bcl-2, anti-Mcl-1, anti-Bcl-xL, anti-BIM, anti-Bax, anti-CD90, anti-CD96, anti-OX40, anti-MIP1a, anti-MIP1b, anti-IL4, anti-IL5, anti-IL17A, anti-IL6, anti-MCP1, anti-CD112, anti-CDD115, anti-CD34, anti-CD11b, anti-CD11a, anti-CD141, anti-CD105, anti-CD1a, anti-CD1c, anti-CD235a, anti-YTHDF2, anti-CD41a, anti-CD71, anti-Siglec7, anti-CD30, anti-CD61, anti-RANTES, anti-MCT-1, anti-MCT4, anti-CD80, anti-CD83, anti-CD86, anti-KIR3DL1, anti-KIR3DL2, anti-KIR2DL1, anti-NKG2C, anti-CD94, anti-NRF2, anti-CDT1, anti-pRB, anti-CyclinB1, anti-BrU, and any combination thereof.

119. The panel of claim 117 or 118, wherein at least two conjugated antibodies of the plurality of conjugated antibodies are tagged with distinct metal tags having different isotopes of a single element.

120. The panel of any one of claims 117-119, wherein the panel is contained in a kit.

121. The panel of claim 120, wherein the kit further comprises a calibration material comprising a known quantity of an isotope present in a metal tag of an antibody of the plurality of conjugated antibodies.

122. A barcoding system comprising: a plurality of barcoding reagents, wherein each of the plurality of barcoding reagents comprises one of a plurality of combinations of metal tags of any one of claims 1-70 and wherein each combination of metal tags is associated with a target antibody.

123. The barcoding system of claim 122, wherein the plurality of combinations of metal tags of the barcoding system is distinct from a metal-tagged antibody that targets an analyte.

124. The barcoding system of claim 122 or 123, wherein each of the plurality of barcoding reagents is contained within a container.

125. The barcoding system of claim 124, wherein the container is a hermetically sealed container.

126. The barcoding system of any one of claims 122-125, wherein the barcoding reagent is a bead.

127. The barcoding system of any one of claims 122-126, wherein each metal tag of the combination of metal tags is distinguishable through elemental analysis.

128. The barcoding system of any one of claims 122-127, wherein the target analyte is present on a cell.

129. The barcoding system of any one of claims 122-128, wherein the cell is a live cell.

130. A method of interrogating or analyzing a sample comprising a cell with an elemental analyzer comprising mixing the sample with the population of metal-tagged probes of any one of claims 71-76 to barcode the cell.

131. The method of claim 130, wherein the elemental analyzer is a mass cytometer and the method comprises performing mass cytometry.

132. The method of claim 130 or 131 comprising:
a. labeling the sample with a population of metal-tagged probes, wherein the population of metal-tagged probes bind to an antigen on a cell and barcode the cell; and
b. detecting the barcoded cell based on the presence of the metal-tagged probe bound to the cell.

133. The method of any one of claims 130-132, wherein the cell is a live cell.

134. The method of claim 131 or 132, wherein the cell is detected at a level of increased sensitivity compared to a metal-tagged probe that does not comprise a metal tag of the population of metal tags of claims 53 32, and/or 42.

135. The method of any one of claims 130-134, wherein the method further comprises
c. labeling the sample with a population of metal-tagged probes, wherein the population of metal tagged probes bind to at least one analyte on a cell that is different from the antigen of the barcoded cell bound by the metal-tagged probes of step (b); and
d. detecting the at least one analyte on the barcoded cell.

136. A method of using a population of metal-tagged probes of any one of claims 71-76 to stain cells in a tissue section sample.
